(19) United States
(12) Patent Application Publication
(10) Pub. No.: US 2025/0034158 A1
(43) Pub. Date: Jan. 30, 2025

(54) TEXAPHYRIN DERIVATIVES FOR MANGANESE CHEMOTHERAPY, PHOTOACOUSTIC IMAGING, AND PHOTOTHERMAL THERAPY

(71) Applicant: BOARD OF REGENTS, THE UNIVERSITY OF TEXAS SYSTEM, Austin, TX (US)

(72) Inventors: Jonathan L. SESSLER, Austin, TX (US); Adam C. SEDGWICK, Austin, TX (US); Gregory THIABAUD, Austin, TX (US); Jonathan ARAMBULA, Austin, TX (US)

(21) Appl. No.: 18/041,470

(22) PCT Filed: Aug. 14, 2021
(86) PCT No.: PCT/US21/46049

§ 371 Date: Feb. 13, 2023

Related U.S. Application Data
(60) Provisional application No. 63/066,001, filed on Aug. 14, 2020.

(51) Int. Cl. C07D 487/22; A61K 31/555; A61K 41/00; A61K 49/22

(52) U.S. Cl. CPC C07D 487/22; A61K 31/555; A61K 41/0052; A61K 49/221

(57) ABSTRACT

The present disclosure relates to manganese containing texaphyrin compounds of the formula (I). Wherein the variables are as described herein. The present disclosure also provides pharmaceutical compositions of the compounds. Also, provided herein are methods of using the compounds in the treatment of cancer including platinum resistant cancer.

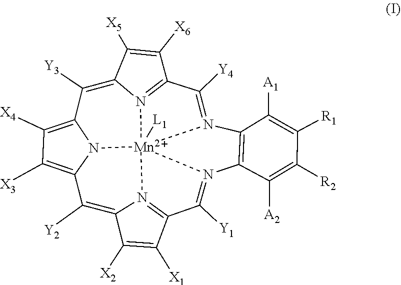

This application claims the benefit of priority to U.S. Provisional Application No. 63/066,001, the entire contents of which are hereby incorporated by reference.

This invention was made with government support under Grant no. R01 CA068682 awarded by the National Institutes of Health. The government has certain rights in the invention.

### BACKGROUND

### 1. Field

The present disclosure relates generally to the fields of medicine, pharmaceutical agents, and chemotherapeutics. The present disclosure relates to texaphyrin conjugates and compositions, which can be used to treat cancer.

### 2. Description of Related Art

Metallotexaphyrins are a class of expanded porphyrins that have been shown to accumulate in primary and metastatic tumors. Moreover, metallotexaphyrins have intrinsic anticancer activity through redox activity centered on the macrocyclic ligand. The texaphyrin core has enabled the formation of stable metal complexes with lanthanide ions and transition metal ions, which then can be used to enhance the contrast of MR images. Previously, it has been shown Gd(III)-texaphyrin-platinum(IV) conjugates are capable of overcoming platinum resistance by localizing to solid tumors, promoting enhanced cancer cell uptake, and reactivating p53 in platinum resistant models. Side-by-side comparative studies of these Pt(IV) conjugates to clinically approved platinum(II) agents and previously reported platinum(II)-texaphyrin conjugates demonstrate that these Pt(IV) conjugates are more stable against hydrolysis, nucleophilic attack, and have displayed high potent antiproliferative activity in vitro against human and mouse cell cancer lines as well as be more efficacious in subcutaneous mouse models involving both cell-derived xenografts and platinum resistant patient-derived xenografts.

Unfortunately, Gd-based molecules are contraindicated for use in renally compromised patients and repeat usage has been shown to lead to the buildup of toxic Gd(III) ions resulting in serious toxicity issues including death. These concerns regarding toxicity have led to certain Gd-based molecules being withdrawn from the market. Manganese (Mn) is an essential trace nutritional element easily processed by the human body with clearance via endogenous mechanisms thus making manganese-based metallotexaphyrin (MMn)-drug conjugates an attractive alternative to their gadolinium congeners. All reported texaphyrins are known to absorb light well in the >700 nm spectral region where tissues are mostly transparent. These are attractive properties for both photothermal therapy (PTT) and photoacoustic imaging (PAI) of solid tumors in vivo. PTT and PAI are techniques that involve the non-radiative conversion of light energy into heat (PTT) or sound energy, respectively. PTT is a highly effective and non-invasive experimental cancer therapy that uses photo-irradiation to increase the temperature of a tumor and its surrounding environment. PAI, on the other hand, uses photons to create agent-induced cavitation effects that can be monitored by ultrasound instrumentation. While this technique is still in an early research stage, sensitizer-induced PAI could be used to overcome the limitations of traditional optical agents by providing tomographic imaging capabilities with high spatial resolution (within hundreds of microns) at large penetration depths (up to centimeters), while ideally allowing the intraoperative delineation of tumor margins. Despite demonstrating promise in animal models, many current PTT/PAI agents rely on combined nanomaterial/nanocarrier systems. This complicates their use and results in known off-target toxicity effects. Moreover, most systems to date have relied on the use of light activation at wavelengths that do not penetrate human tissues well.

Therefore, given the limitations with current PTT/PAI agents, there remains a need to develop new compounds which may be useful as PTT/PAI agents. In particular attention is the development of PTT/PAI agents that may also be used as therapeutic agents.

### SUMMARY

In some aspects, the present disclosure provides compounds that may be used as PTT/PAI agents as well as contain a metal therapeutic center. In some embodiments, the compounds of the formula:

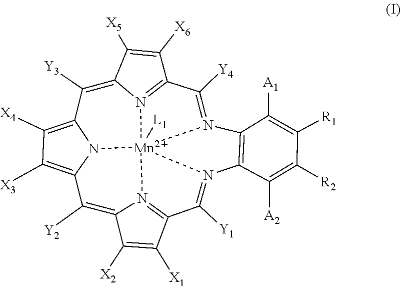

wherein:
R₁ and R₂ are each independently hydroxy, alkoxy_{(C≤12)}, substituted alkoxy_{(C≤12)},

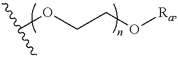

wherein n is 1-8 and Rₐ is hydrogen, alkyl_{(C≤6)}, or substituted alkyl_{(C≤6)}, or

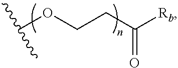

wherein m is 1-8 and R_{b} is hydroxy, alkoxy_{(C≤6)}, substituted alkoxy_{(C≤6)}, alkylamino_{(C≤6)}, substituted alkylamino_{(C≤6)}, dialkylamino_{(C≤6)}, substituted dialkylamino_{(C≤6)}, or a sugar moiety;
A₁ and A₂ are each hydrogen, halo, hydroxy, alkyl_{(C≤8)}, substituted alkyl_{(C≤8)}, aryl_{(C≤8)}, or substituted aryl_{(C≤8)};
Y₁, Y₂, Y₃, and Y₄ are each independently hydrogen, halo, hydroxy, alkyl_{(C≤8)}, or substituted alkyl_{(C≤8)};
X₁, X₂, X₃, X₄, X₅, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof, or a platinum(IV) chelating group; provided at least one of X₁-X₆ is a platinum(IV) chelating group, wherein the platinum(IV) chelating group is further defined as:

-A₃-Y₅-A₄-R_{c}

wherein:
A₃ and A₄ are each independently selected from alkanediyl_{(C≤8)}, substituted alkanediyl_{(C≤8)}, or

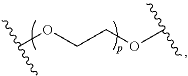

wherein p is 1-8;
Y₅ is —C(O)NR_{d}— or —NR_{d}C(O)—;
   R_{d} is hydrogen, alkyl_{(C≤6)}, or substituted alkyl_{(C≤6)};
R_{c} is a group of the formula:

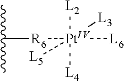

wherein:
   R₆ is carboxy;
   L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤18)};
   L₆ is aqua, ammonia, nitrate, sulfate, halide, hydroxide, phosphate, or glucose-6-phosphate,
   alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
L₁ is a monovalent anionic group;
   or a pharmaceutically acceptable salt thereof.

In some embodiments, the compounds are further defined as:

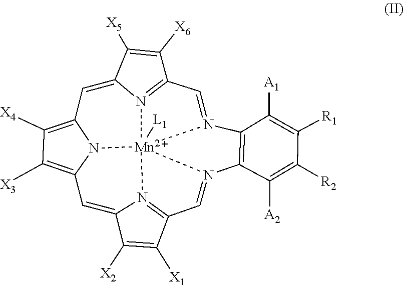

wherein:
R₁ and R₂ are each independently hydroxy, alkoxy_{(C≤12)}, substituted alkoxy_{(C≤12)},

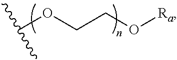

wherein n is 1-8 and Rₐ is hydrogen, alkyl_{(C≤6)}, or substituted alkyl_{(C≤6)}, or

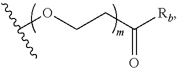

wherein m is 1-8 and R_{b} is hydroxy, alkoxy_{(C≤6)}, substituted alkoxy_{(C≤6)}, alkylamino_{(C≤6)}, substituted alkylamino_{(C≤6)}, dialkylamino_{(C≤6)}, substituted dialkylamino_{(C≤6)}, or a sugar moiety;
A₁ and A₂ are each hydrogen, halo, hydroxy, alkyl_{(C≤8)}, substituted alkyl_{(C≤8)}, aryl_{(C≤8)}, or substituted aryl_{(C≤8)};
X₁, X₂, X₃, X₄, X₅, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof, or a platinum(IV) chelating group; provided at least one of X₁-X₆ is a platinum(IV) chelating group, wherein the platinum(IV) chelating group is further defined as:

-A₃-Y₅-A₄-R_{c}
wherein:
   A₃ and A₄ are each independently selected from alkanediyl_{(C≤8)}, substituted alkanediyl_{(C≤8)}, or

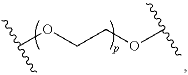

wherein p is 1-8;
Y₅ is —C(O)NR_{d}— or —NR_{d}C(O)—;
   R_{d} is hydrogen, alkyl_{(C≤6)}, or substituted alkyl_{(C≤6)};
R_{c} is a group of the formula:

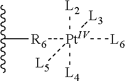

wherein:
   R₆ is carboxy;
   L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤18)};
   L₆ is aqua, ammonia, nitrate, sulfate, halide, hydroxide, phosphate, or glucose-6-phosphate,
   alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
L₁ is a monovalent anionic group;
   or a pharmaceutically acceptable salt thereof.

In some embodiments, the compounds are further defined as:

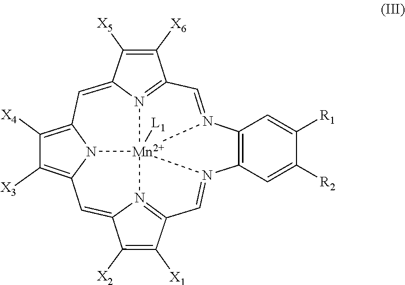

wherein:
R₁ and R₂ are each independently hydroxy, alkoxy_{(C≤12)}, substituted alkoxy_{(C≤12)},

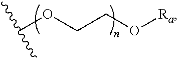

wherein n is 1-8 and Rₐ is hydrogen, alkyl_{(C≤6)}, or substituted alkyl_{(C≤6)}, or

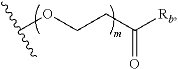

wherein m is 1-8 and R_{b} is hydroxy, alkoxy_{(C≤6)}, substituted alkoxy_{(C≤6)}, alkylamino_{(C≤6)}, substituted alkylamino_{(C≤6)}, dialkylamino_{(C≤6)}, substituted dialkylamino_{(C≤6)}, or a sugar moiety;
X₁, X₂, X₃, X₄, X₅, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof, or a platinum(IV) chelating group; provided at least one of X₁-X₆ is a platinum(IV) chelating group, wherein the platinum(IV) chelating group is further defined as:

-A₃-Y₅-A₄-R_{c}

wherein:
A₃ and A₄ are each independently selected from alkanediyl_{(C≤8)}, substituted alkanediyl_{(C≤8)}, or

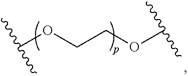

wherein p is 1-8;
Y₅ is —C(O)NR_{d}— or —NR_{d}C(O)—;
   R_{d} is hydrogen, alkyl_{(C≤6)}, or substituted alkyl_{(C≤6)};
R_{c} is a group of the formula:

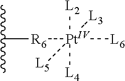

wherein:
   R₆ is carboxy;
   L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤18)};
   L₆ is aqua, ammonia, nitrate, sulfate, halide, hydroxide, phosphate, or glucose-6-phosphate,
   alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
L₁ is a monovalent anionic group;
   or a pharmaceutically acceptable salt thereof.

In some embodiments, the compounds are further defined as:

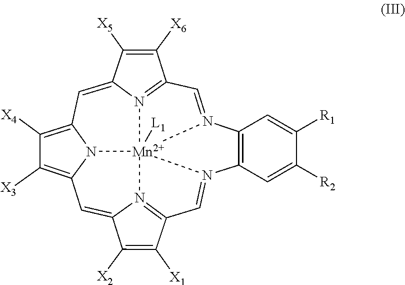

wherein:
R₁ and R₂ are each independently hydroxy, alkoxy_{(C≤12)}, substituted alkoxy_{(C≤12)},

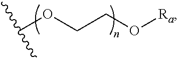

wherein n is 1-8 and Rₐ is hydrogen, alkyl_{(C≤6)}, or substituted alkyl_{(C≤6)};
X₁, X₂, X₃, X₄, X₅, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof, or a platinum(IV) chelating group; provided at least one of X₁-X₆ is a platinum(IV) chelating group, wherein the platinum(IV) chelating group is further defined as:

-A₃-Y₅-A₄-R_{c}

wherein:
A₃ and A₄ are each independently selected from alkanediyl_{(C≤8)}, substituted alkanediyl_{(C≤8)}, or

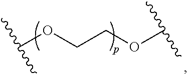

wherein p is 1-8;
Y₅ is —C(O)NR_{d}— or —NR_{d}C(O)—;
   R_{d} is hydrogen, alkyl_{(C≤6)}, or substituted alkyl_{(C≤6)};
R_{c} is a group of the formula:

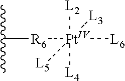

wherein:
   R₆ is carboxy;
   L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤18)};
   L₆ is aqua, ammonia, nitrate, sulfate, halide, hydroxide, phosphate, or glucose-6-phosphate,
   alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤12)}, arylcarboxylate_{(C≤12)}, aryldicarboxylate_{(C≤18)}, or a substituted version of any of these groups;
L₁ is a monovalent anionic group;
   or a pharmaceutically acceptable salt thereof.

In some embodiments, the compounds are further defined as:

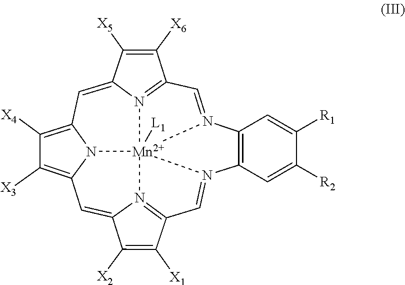

wherein:
R₁ and R₂ are each independently hydroxy, alkoxy_{(C≤12)}, substituted alkoxy_{(C≤12)},

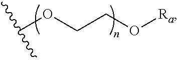

wherein n is 1-8 and Rₐ is hydrogen, alkyl_{(C≤6)}, or substituted alkyl_{(C≤6)};
X₁, X₃, X₄, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof;
X₂ and X₅ are each independently alkyl_{(C≤8)}, substituted alkyl_{(C≤8)}, a platinum(IV) chelating group; provided either X₂ or X₅ is a platinum(IV) chelating group, wherein the platinum(IV) chelating group is further defined as:

-A₃-Y₅-A₄-R_{c}
wherein:
   A₃ and A₄ are each independently selected from alkanediyl_{(C≤8)}, substituted alkanediyl_{(C≤8)}, or

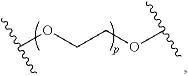

wherein p is 1-8;
Y₅ is —C(O)NR_{d}— or —NR_{d}C(O)—;
   R_{d} is hydrogen, alkyl_{(C≤6)}, or substituted alkyl_{(C≤6)};
R_{c} is a group of the formula:

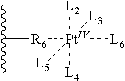

wherein:
   R₆ is carboxy;
   L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤18)};
   L₆ is aqua, ammonia, nitrate, sulfate, halide, hydroxide, phosphate, or glucose-6-phosphate,
   alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups; and
L₁ is a monovalent anionic group;
   or a pharmaceutically acceptable salt thereof.

In some embodiments, the compounds are further defined as:

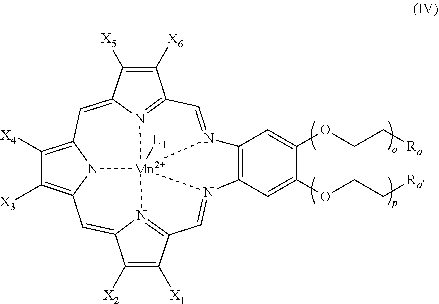

wherein:
Rₐ and Rₐ′ are each independently hydrogen, alkyl_{(C≤6)}, or substituted alkyl_{(C≤6)};
o and p are each independent 1, 2, 3, or 4;
X₁, X₃, X₄, and X₆ are each independently hydrogen, alkyl_{(C≤8)}, cycloalkyl_{(C≤8)}, alkenyl_{(C≤8)}, alkynyl_{(C≤8)}, aryl(c≤₈), heteroaryl_{(C≤8)}, heterocycloalkyl_{(C≤8)}, or a substituted version thereof;
X₂ and X₅ are each independently alkyl_{(C≤8)}, substituted alkyl_{(C≤8)}, a platinum(IV) chelating group; provided either X₂ or X₅ is a platinum(IV) chelating group, wherein the platinum(IV) chelating group is further defined as:

-A₃-Y₅-A₄-R_{c}
wherein:
   A₃ and A₄ are each independently selected from alkanediyl_{(C≤8)}, substituted alkanediyl_{(C≤8)}, or

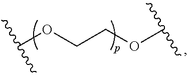

wherein p is 1-8;
Y₅ is —C(O)NR_{d}— or —NR_{d}C(O)—;
   R_{d} is hydrogen, alkyl_{(C≤6)}, or substituted alkyl_{(C≤6)};
R_{c} is a group of the formula:

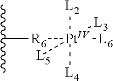

wherein:
   R₆ is carboxy;
   L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤8)};
   L₆ is aqua, ammonia, nitrate, sulfate, halide, hydroxide, phosphate, or glucose-6-phosphate,
   alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups; and
L₁ is a monovalent anionic group;
   or a pharmaceutically acceptable salt thereof.

In some embodiments, Y₁ is hydrogen. In some embodiments, Y₂ is hydrogen. In some embodiments, Y₃ is hydrogen. In some embodiments, Y₄ is hydrogen. In some embodiments, A₁ is hydrogen. In some embodiments, A₂ is hydrogen.

In some embodiments, R₁ is

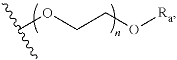

wherein n is 1-8 and Rₐ is hydrogen, alkyl_{(C≤6)}, or substituted alkyl_{(C≤6)}. In some embodiments, n is 1, 2, 3, or 4. In some embodiments, n is 2, 3, or 4. In some embodiments, n is 3 or 4. In some embodiments, n is 3. In some embodiments, Rₐ is alkyl_{(C≤6)} such as methyl.

In some embodiments, R₂ is

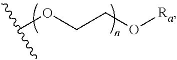

wherein n is 1-8 and Rₐ is hydrogen, alkyl_{(C≤6)}, or substituted alkyl_{(C≤6)}. In some embodiments, n is 1, 2, 3, or 4. In some embodiments, n is 2, 3, or 4. In some embodiments, n is 3 or 4. In some embodiments, n is 3. In some embodiments, Rₐ is alkyl_{(C≤6)} such as methyl.

In some embodiments, X₁ is alkyl_{(C≤8)} or substituted alkyl_{(C≤8)}. In some embodiments, X₁ is alkyl_{(C≤8)} such as methyl. In some embodiments, X₂ is a platinum(IV) chelating group. In some embodiments, A₃ is alkanediyl_{(C≤8)} such as propylene. In other embodiments, A₃ is

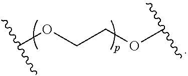

In some embodiments, Y₅ is —NR_{d}C(O)—. In some embodiments, Y₅ is hydrogen. In some embodiments, A₄ is alkanediyl_{(C≤8)} such as ethylene. In other embodiments, A₄ is

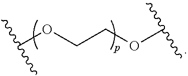

In some embodiments, L₂ is halide such as chloride. In other embodiments, L₂ is ammonia. In other embodiments, L₂ and L₃ are taken together and are diaminocycloalkane_{(C≤18)} or substituted diaminocycloalkane_{(C≤18)}. In some embodiments, L₂ and L₃ are taken together and are diaminocycloalkane_{(C≤18)} such as diaminocyclohexane. In other embodiments, L₂ and L₃ are taken together and are alkyldicarboxylate_{(C≤18)} or substituted alkyldicarboxylate_{(C≤18)}. In some embodiments, L₂ and L₃ are taken together and are alkyldicarboxylate_{(C≤18)} such as oxalic acid. In other embodiments, L₃ is halide such as chloride. In other embodiments, L₃ is ammonia. In some embodiments, L₄ is halide such as chloride. In other embodiments, L₄ is ammonia. In other embodiments, L₄ and L₅ are taken together and are diaminocycloalkane_{(C≤18)} or substituted diaminocycloalkane_{(C≤18)}. In some embodiments, L₄ and L₅ are taken together and are diaminocycloalkane_{(C≤18)} such as diaminocyclohexane. In other embodiments, L₄ and L₅ are taken together and are alkyldicarboxylate_{(C≤18)} or substituted alkyldicarboxylate_{(C≤18)}. In some embodiments, L₄ and L₅ are taken together and are alkyldicarboxylate_{(C≤18)} such as oxalic acid. In other embodiments, L₅ is halide such as chloride. In other embodiments, L₅ is ammonia. In some embodiments, L₆ is hydroxy. In other embodiments, L₆ is alkylcarboxylate_{(C≤12)} or substituted alkylcarboxylate_{(C≤12)}. In some embodiments, L₆ is alkylcarboxylate_{(C≤12)} such as acetate. In other embodiments, L₆ is halo such as chloro.

In other embodiments, X₂ is alkyl_{(C≤8)} or substituted alkyl_{(C≤8)}. In some embodiments, X₂ is substituted alkyl_{(C≤8)} such as 3-hydroxypropyl.

In some embodiments, X₃ is alkyl_{(C≤8)} or substituted alkyl_{(C≤8)}. In some embodiments, X₃ is alkyl_{(C≤8)} such as ethyl. In some embodiments, X₄ is alkyl_{(C≤8)} or substituted alkyl_{(C≤8)}. In some embodiments, X₄ is alkyl_{(C≤8)} such as ethyl.

In some embodiments, X₅ is a platinum(IV) chelating group. In some embodiments, A₃ is alkanediyl_{(C≤8)} such as propylene. In other embodiments, A₃ is

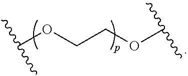

In some embodiments, Y₅ is —NR_{d}C(O)—. In some embodiments, Y₅ is hydrogen. In some embodiments, A₄ is alkanediyl_{(C≤8)} such as ethylene. In other embodiments, A₄ is

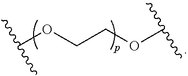

In some embodiments, L₂ is halide such as chloride. In other embodiments, L₂ is ammonia. In other embodiments, L₂ and L₃ are taken together and are diaminocycloalkane_{(C≤18)} or substituted diaminocycloalkane_{(C≤18)}. In some embodiments, L₂ and L₃ are taken together and are diaminocycloalkane_{(C≤18)} such as diaminocyclohexane. In other embodiments, L₂ and L₃ are taken together and are alkyldicarboxylate_{(C≤18)} or substituted alkyldicarboxylate_{(C≤18)}. In some embodiments, L₂ and L₃ are taken together and are alkyldicarboxylate_{(C≤18)} such as oxalic acid. In other embodiments, L₃ is halide such as chloride. In other embodiments, L₃ is ammonia. In some embodiments, L₄ is halide such as chloride. In other embodiments, L₄ is ammonia. In other embodiments, L₄ and L₅ are taken together and are diaminocycloalkane_{(C≤18)} or substituted diaminocycloalkane_{(C≤18)}. In some embodiments, L₄ and L₅ are taken together and are diaminocycloalkane_{(C≤18)} such as diaminocyclohexane. In other embodiments, L₄ and L₅ are taken together and are alkyldicarboxylate_{(C≤18)} or substituted alkyldicarboxylate_{(C≤18)}. In some embodiments, L₄ and L₅ are taken together and are alkyldicarboxylate_{(C≤18)} such as oxalic acid. In other embodiments, L₅ is halide such as chloride. In other embodiments, L₅ is ammonia. In some embodiments, L₆ is hydroxy. In other embodiments, L₆ is alkylcarboxylate_{(C≤12)} or substituted alkylcarboxylate_{(C≤12)}. In some embodiments, L₆ is alkylcarboxylate_{(C≤12)} such as acetate. In other embodiments, L₆ is halo such as chloro. In other embodiments, X₅ is alkyl_{(C≤8)} or substituted alkyl_{(C≤8)}. In some embodiments, X₅ is substituted alkyl_{(C≤8)} such as 3-hydroxypropyl.

In some embodiments, X₆ is alkyl_{(C≤8)} or substituted alkyl_{(C≤8)}. In some embodiments, X₆ is alkyl_{(C≤8)} such as methyl. In some embodiments, L₁ is nitrate. In other embodiments, L₁ is alkylcarboxylate_{(C≤12)} or substituted alkylcarboxylate_{(C≤12)}. In some embodiments, L₁ is alkylcarboxylate_{(C≤12)} such as acetate.

In some embodiments, the compounds are further defined as:

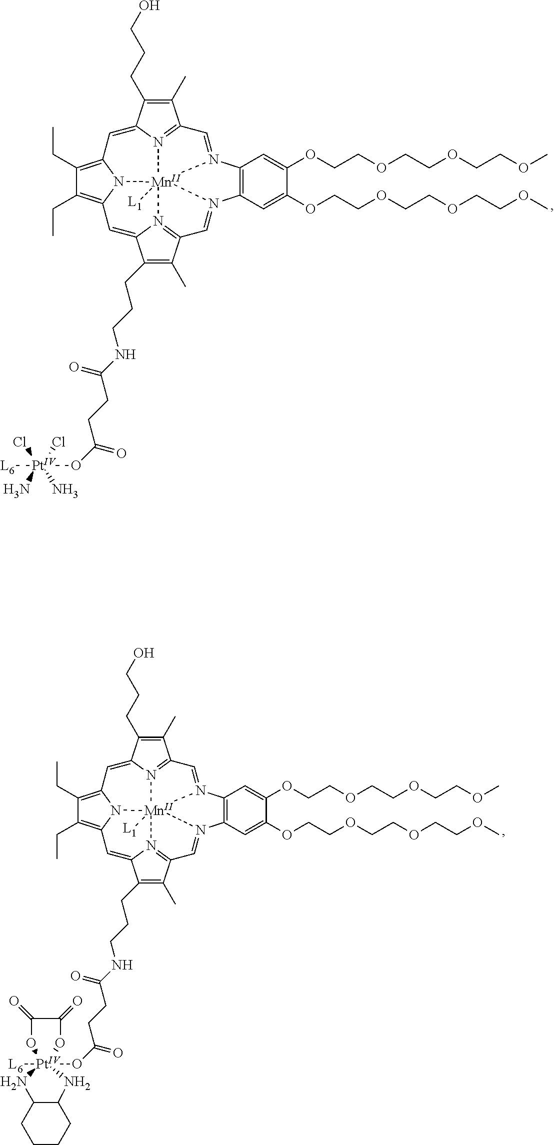

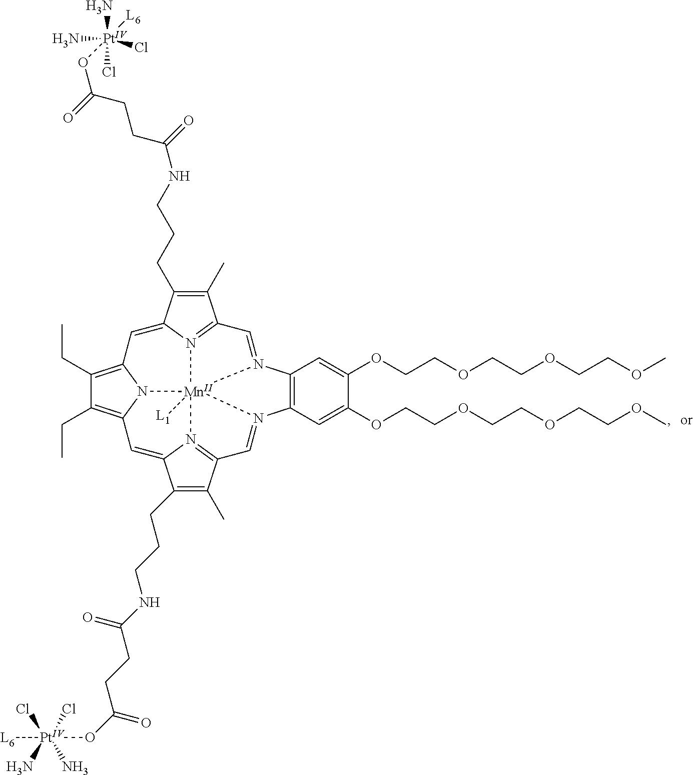

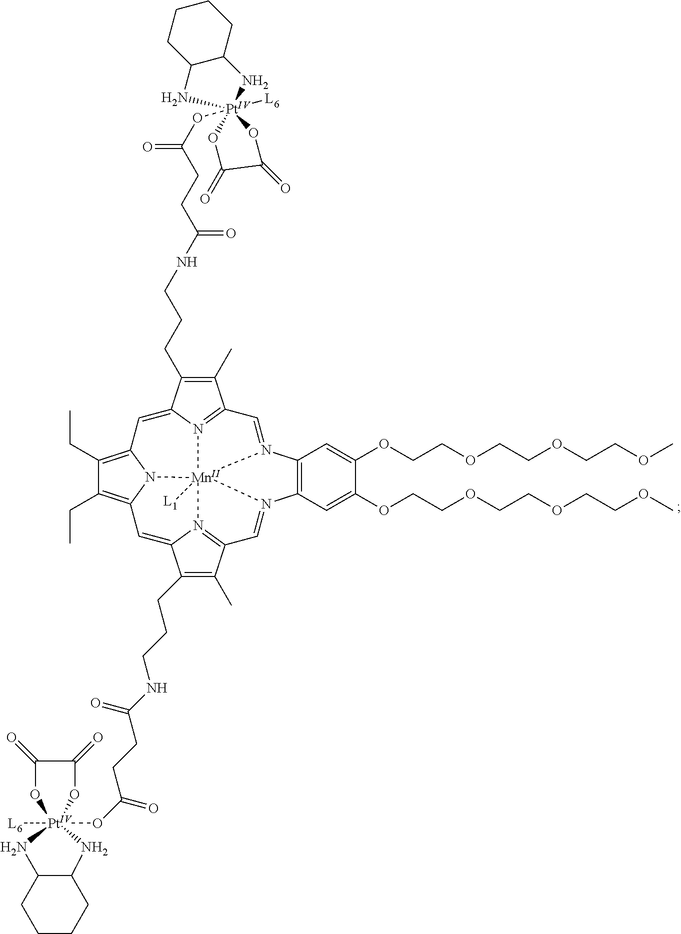

wherein:
L₁ is a monovalent anionic group; and
each L₆ is aqua, ammonia, nitrate, sulfate, halide, hydroxide, phosphate, or glucose-6-phosphate,
alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
   or a pharmaceutically acceptable salt thereof.

In some embodiments, the compounds are further defined as:

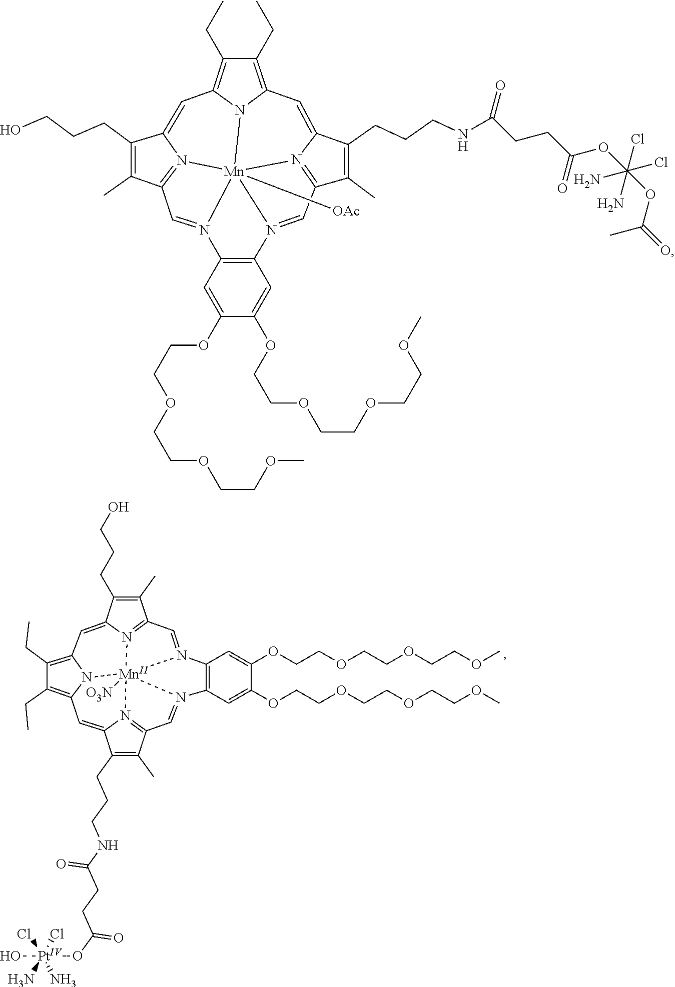

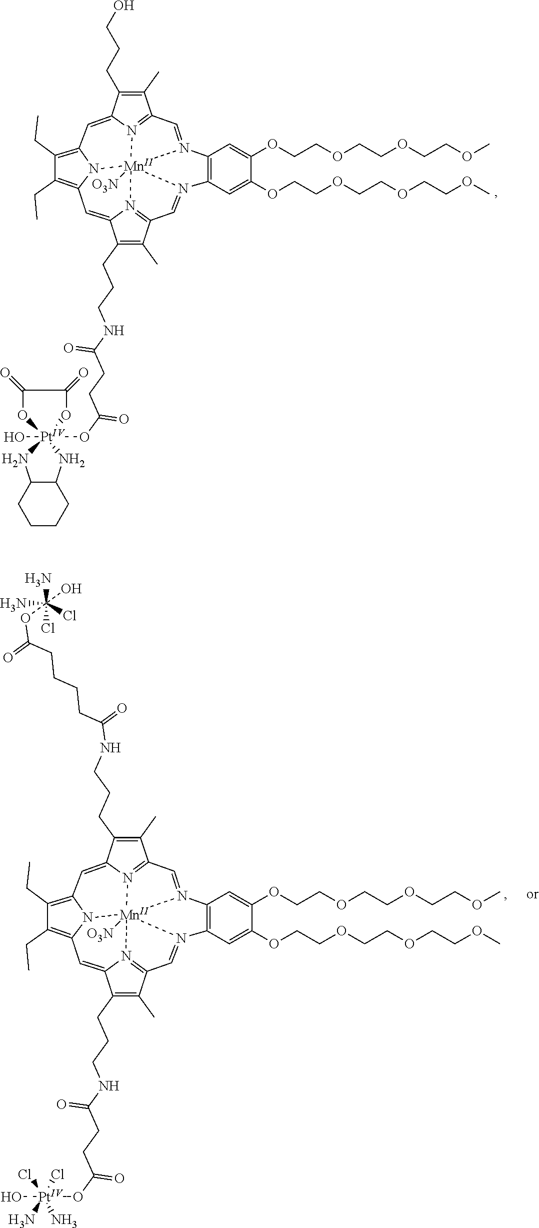

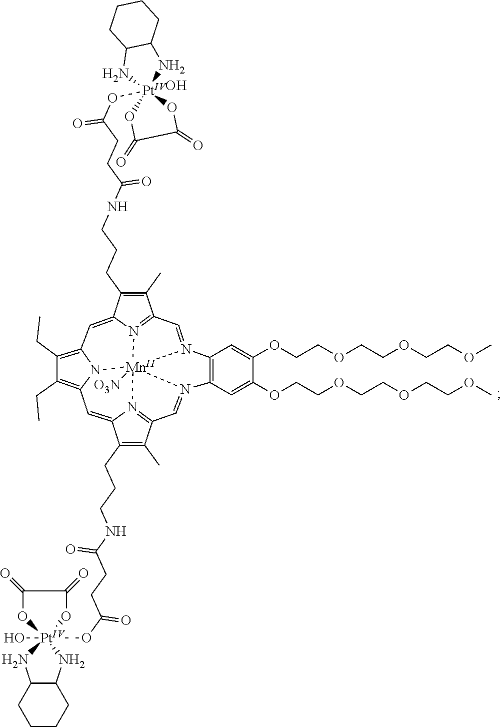

or a pharmaceutically acceptable salt thereof.

In still another aspect, the present disclosure provides pharmaceutical compositions comprising:
(A) a compound described herein; and
(B) an excipient.

In some embodiments, the pharmaceutical compositions are formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. In some embodiments, the pharmaceutical compositions are formulated for oral administration or administration via injection. In some embodiments, the administration via injection is intraarterial administration, intraperitoneal administration, intravenous administration, or subcutaneous administration. In some embodiments, the pharmaceutical compositions are formulated as a unit dose.

In still yet another aspect, the present disclosure provides methods of treating a disease comprising administering a therapeutically effective amount of a compound or pharmaceutical composition described herein to a patient in need thereof. In some embodiments, the disease is cancer. In some embodiments, the cancer is a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the cancer is resistant to one or more platinum chemotherapeutic agents. In some embodiments, the cancer is resistant to cisplatin or oxaliplatin such as resistant to cisplatin and oxaliplatin. In some embodiments, the cancer is ovarian cancer, lung cancer, breast cancer, endometrial cancer, brain cancer, skin cancer, head and neck cancer, or colorectal cancer. In some embodiments, the methods further comprise administering a second therapeutic agent such as a second chemotherapeutic agent, surgery, photodynamic therapy, sonodynamic therapy, radiotherapy, or immunotherapy.

In still yet another aspect, the present disclosure provides methods of obtaining an image of a patient comprising administering to the patient an effective amount of a compound or a pharmaceutical composition comprising a compound of the formula:

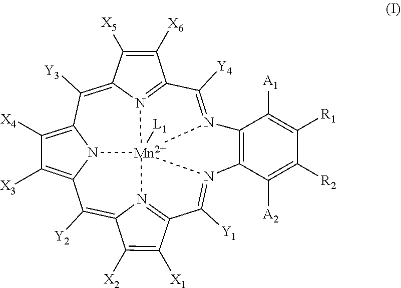

wherein:
R₁ and R₂ are each independently hydroxy, alkoxy_{(C≤12)}, substituted alkoxy_{(C≤12)},

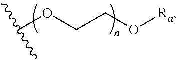

wherein n is 1-8 and Rₐ is hydrogen, alkyl_{(C≤6)}, or substituted alkyl_{(C≤6)}, or

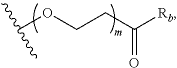

wherein m is 1-8 and R_{b} is hydroxy, alkoxy_{(C≤6)}, substituted alkoxy_{(C≤6)}, alkylamino_{(C≤6)}, substituted alkylamino_{(C≤6)}, dialkylamino_{(C≤6)}, substituted dialkylamino_{(C≤6)}, or a sugar moiety;
A₁ and A₂ are each hydrogen, halo, hydroxy, alkyl_{(C≤8)}, substituted alkyl_{(C≤8)}, aryl_{(C≤8)}, or substituted aryl_{(C≤8)};
Y₁, Y₂, Y₃, and Y₄ are each independently hydrogen, halo, hydroxy, alkyl_{(C≤8)}, or substituted alkyl_{(C≤8)};
X₁, X₂, X₃, X₄, X₅, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof, or a platinum(IV) chelating group; wherein the platinum(IV) chelating group is further defined as:

-A₃-Y₅-A₄-R_{c}
wherein:
   A₃ and A₄ are each independently selected from alkanediyl_{(C≤8)}, substituted alkanediyl_{(C≤8)}, or

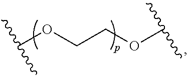

wherein p is 1-8;
Y₅ is —C(O)NR_{d}— or —NR_{d}C(O)—;
   R_{d} is hydrogen, alkyl_{(C≤6)}, or substituted alkyl_{(C≤6)};
R_{c} is a group of the formula:

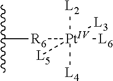

wherein:
   R₆ is carboxy;
   L₂-L₅ are each independently selected from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤8)};
   L₆ is aqua, ammonia, nitrate, sulfate, halide, hydroxide, phosphate, or glucose-6-phosphate,
   alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
L₁ is a monovalent anionic group;
   and imaging the patient to obtain the image of the patient.

In some embodiments, A₁ and A₂ are hydrogen. In some embodiments, Y₁, Y₂, Y₃, and Y₄ are hydrogen. In some embodiments, X₁ and X₆ are alkyl_{(C≤6)} such as methyl. In some embodiments, X₃ and X₄ are alkyl_{(C≤6)} such as ethyl. In some embodiments, X₂ and X₅ are substituted alkyl_{(C≤6)} such as 3-hydroxypropyl. In some embodiments, the compound is further defined as:

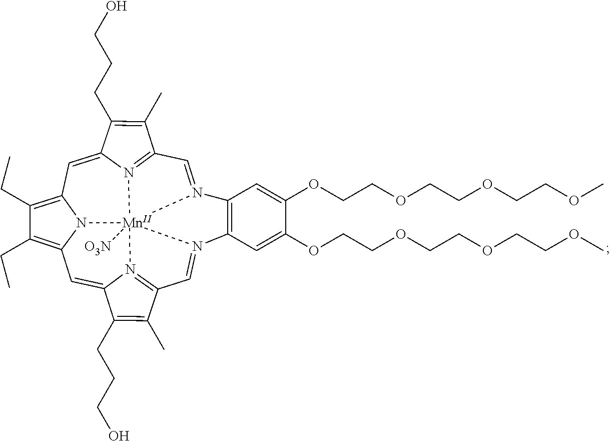

or a pharmaceutically acceptable salt thereof.

In some embodiments, the patient is imaged using laser pulses. In some embodiments, the patient is imaged using a wavelength from about 500 nm to about 1300 nm. In some embodiments, the wavelength is in the near-IR or IR range. In some embodiments, the wavelength is in the near IR such as from about 650 nm to about 780 nm. In some embodiments, the imaging is photoacoustic imaging. In some embodiments, the photoacoustic imaging is photoacoustic tomography. In other embodiments, the photoacoustic imaging is photoacoustic microscopy. In other embodiments, the patient is imaged using magnetic resonance imaging. In some embodiments, the method images a tumor such as a solid tumor. In some embodiments, the solid tumor is ovarian cancer, lung cancer, breast cancer, endometrial cancer, brain cancer, skin cancer, head and neck cancer, or colorectal cancer.

In yet another aspect, the present disclosure provides methods of treating a patient comprising administering a compound of the formula:

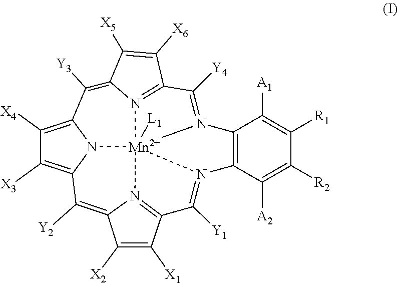

wherein:
R₁ and R₂ are each independently hydroxy, alkoxy_{(C≤12)}, substituted alkoxy_{(C≤12)},

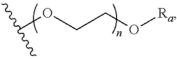

wherein n is 1-8 and Rₐ is hydrogen, alkyl_{(C≤6)}, or substituted alkyl_{(C≤6)}, or

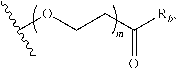

wherein m is 1-8 and R_{b} is hydroxy, alkoxy_{(C≤6)}, substituted alkoxy_{(C≤6)}, alkylamino_{(C≤6)}, substituted alkylamino_{(C≤6)}, dialkylamino_{(C≤6)}, substituted dialkylamino_{(C≤6)}, or a sugar moiety;
A₁ and A₂ are each hydrogen, halo, hydroxy, alkyl_{(C≤8)}, substituted alkyl_{(C≤8)}, aryl_{(C≤8)}, or substituted aryl_{(C≤8)};
Y₁, Y₂, Y₃, and Y₄ are each independently hydrogen, halo, hydroxy, alkyl_{(C≤8)}, or substituted alkyl_{(C≤8)};
X₁, X₂, X₃, X₄, X₅, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof, or a platinum(IV) chelating group; wherein the platinum(IV) chelating group is further defined as:

-A₃-Y₅-A₄-R_{c}
wherein:
   A₃ and A₄ are each independently selected from alkanediyl_{(C≤8)}, substituted alkanediyl_{(C≤8)}, or

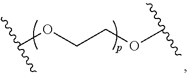

wherein p is 1-8;
Y₅ is —C(O)NR_{d}— or —NR_{d}C(O)—;
   R_{d} is hydrogen, alkyl_{(C≤6)}, or substituted alkyl_{(C≤6)};
R_{c} is a group of the formula:

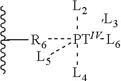

wherein:
   R₆ is carboxy;
   L₂-L₅ are each independently selected from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤8)};
   L₆ is aqua, ammonia, nitrate, sulfate, halide, hydroxide, phosphate, or glucose-6-phosphate,
   alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
L₁ is a monovalent anionic group;
   to a patient in need thereof and exposing the patient to an electromagnetic radiation.

In some embodiments, A₁ and A₂ are hydrogen. In some embodiments, Y₁, Y₂, Y₃, and Y₄ are hydrogen. In some embodiments, X₁ and X₆ are alkyl_{(C≤6)} such as methyl. In some embodiments, X₃ and X₄ are alkyl_{(C≤6)} such as ethyl. In some embodiments, X₂ and X₅ are substituted alkyl_{(C≤6)} such as 3-hydroxypropyl. In some embodiments, the compound is further defined as:

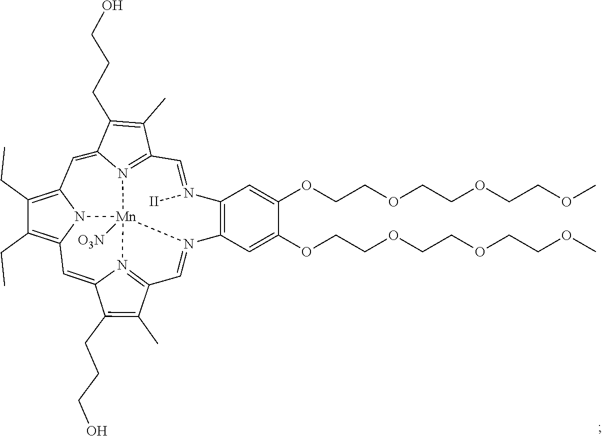

or a pharmaceutically acceptable salt thereof.

In some embodiments, the patient is imaged using laser pulses. In some embodiments, the patient is imaged using a wavelength from about 500 nm to about 1300 nm. In some embodiments, the wavelength is in the near-IR or IR range. In some embodiments, the wavelength is in the near IR such as from about 650 nm to about 780 nm. In some embodiments, the patient is a mammal such as a human.

It is contemplated that any method or composition described herein can be implemented with respect to any other method or composition described herein.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a method, composition, kit, or system that “comprises,” “has,” “contains,” or “includes” one or more recited steps or elements possesses those recited steps or elements, but is not limited to possessing only those steps or elements; it may possess (i.e., cover) elements or steps that are not recited. Likewise, an element of a method, composition, kit, or system that “comprises,” “has,” “contains,” or “includes” one or more recited features possesses those features, but is not limited to possessing only those features; it may possess features that are not recited.

Any embodiment of any of the present methods, composition, kit, and systems may consist of or consist essentially of—rather than comprise/include/contain/have—the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a general schematic of PACT system for PA-based in vivo imaging.

FIG. 2 shows the UV-Vis absorbance spectra of the manganese(II) texaphyrin (MMn), and the corresponding gadolinium(III) (MGd) and lutetium(III) (MLu) complexes recorded in PBS buffer, 25 μM, pH=7.40. Also shown is the chemical structure of these metallotexaphyrins. M=Mn (x=1), Gd (x=2) and Lu (x=2), where x is an anion present either as an axial ligand or as a counter anion.

FIG. 3 shows the PA imaging of ICG, MMn, MLu, and MGd at different concentrations, (15.6-500 μM). Photographs of each solution being tested (left) and the corresponding maximum amplitude projection (MAP) PA images (right). Excitation wavelength: MMn 725 nm, MLu 733 nm MGd: 741 nm. Laser fluence: ˜18 mJ/cm².

FIG. 4 shows the photoacoustic intensity of ICG, MLu, MGd and MMn over a range of concentrations (0-500 μM) in doubly distilled water, pH=7.40. Laser fluence on the sample surface ˜18 mJ/cm² giving a laser energy at the output end of the PA probe of 18 mJ. Laser wavelength used for MMn, MGd and MLu was 725 nm, 741 nm and 733 nm, respectively.

FIG. 5 shows the photoacoustic intensities of ICG, MMn, MLu, at different concentrations in doubly distilled water, pH=7.40. Laser wavelengths used for ICG, MMn, and MLu are 800 nm, 725 nm and 733 nm, respectively. Laser fluence on the sample surface ˜18 mJ/cm² giving a laser energy at the output end of the PA probe of about 18 mJ.

FIG. 6 shows the MAP PA images of ICG, MMn and MLu (4-0.25 mM) in doubly distilled water. Laser wavelengths used for ICG, MMn, and MLu are 800 nm, 725 nm and 733 nm, respectively. Laser fluence on the sample surface ˜18 mJ/cm² giving a laser energy at the output end of the PA probe of about 18 mJ.

FIG. 7 shows the UV-Vis absorption spectra of MMn recorded as a function of increasing concentration (0-170 μM) in deionized water.

FIG. 8 shows the change in the absorption of MMn at 725 nm as a function of increasing concentration (0-170 μM in deionized water.

FIG. 9 shows the fluorescence spectra of MLu, MGd and MMn (20 μM) in PBS buffer solution, pH=7.40, hₑₓ=470 nm, slit widths: ex=10 nm, em=20 nm. Note—a shorter excitation wavelength was used to avoid the small Stokes shift at >700 nm excitation. However, a similar trend is observed when longer excitation wavelengths were employed.

FIGS. 10A-10D show the photosensitized singlet oxygen generation by MLu and MMn. Time-dependent UV absorption spectral changes seen for DMSO solutions of (FIG. 10A) 1,3-diphenylisobenzofuran (DPBF) and (FIG. 10B) MMn or (FIG. 10C) MLu upon irradiation at 730 nm (the power of excitation source is 1.2 mW). (FIG. 10D) Plots of the change in UV absorption at 417 nm for the experiments shown in (FIGS. 10A, FIG. 10B, & FIG. 10C), respectively

FIG. 11 shows the photographs of solutions containing ICG, MMn and MGd (left to right) before and after photoirradiation (20 minutes). Fluence: ˜18 mJ/cm². Laser wavelength: MMn=725 nm, MGd=740 nm and ICG=780 nm.

FIG. 12 shows the UV-Vis absorption spectra of ICG (40 μM) in doubly distilled water recorded while subjecting to photoirradiation for 30 minutes. Measurements were taken at 5-minute intervals. Fluence: 20 mJ/cm². Laser wavelength: 780 nm.

FIG. 13 shows the absorption intensity of ICG (40 μM) at 780 nm over time (0-30 mins) in doubly distilled water under the conditions of photoirradiation. Measurements were taken at 5-minute intervals. Fluence: 20 mJ/cm². Laser wavelength: 780 nm.

FIG. 14 shows the UV-Vis absorption spectra of MMn (40 μM) in doubly distilled water recorded while subjecting to photoirradiation for 30 minutes. Measurements were taken at 5-minute intervals. Fluence: 20 mJ/cm². Laser wavelength: 780 nm.

FIG. 15 shows the absorption intensity of MMn (40 μM) at 725 nm over time (0-30 mins) in doubly distilled water under the conditions of photoirradiation. Measurements were taken at 5-minute intervals. Fluence: 20 mJ/cm². Laser wavelength: 725 nm.

FIG. 16 shows the UV-Vis absorption spectra of MGd (40 μM) in doubly distilled water recorded while subjecting to photoirradiation for 30 minutes. Measurements were taken at 5-minute intervals. Fluence: 20 mJ/cm². Laser wavelength: 780 nm.

FIG. 17 shows the absorption intensity of MGd (40 μM) at 740 nm over time (0-30 mins) in doubly distilled water under the conditions of photoirradiation. Measurements were taken at 5-minute intervals. Fluence: 20 mJ/cm². Laser wavelength: 740 nm.

FIGS. 18A-18C show the results of representative intracellular PAI experiments. RAW 264.7 cells viewed under a microscope and MAP photoacoustic image. (FIG. 18A) Control (doubly distilled water) (FIG. 18B) MMn and (FIG. 18C) MGd. PA images of RAW 264.7 cells cultured with MMn and MGd were at a concentration of 500 μM, respectively. Excitation wavelength: Control: 725 nm, MMn: 725 nm and MGd: 741 nm. Laser fluence ˜18 mJ/cm²

FIG. 19 shows the PA imaging with a prostate tumor mouse model showing the normalized PA intensity of a xenograft tumor at different time points. MMn (500 μM, 200 μL) was administered via tail vein injection. Laser wavelength=725 nm. Laser fluence ˜18 mJ/cm². Pre=prior to injection of MMn.

FIG. 20 shows the normalized PA intensity changes over time (0-48 h) at the tumor site after the injection of saline solution (200 μL) into the tail vein of the mouse

FIG. 21 shows the normalized PA intensity changes over time (0-48 h) at the tumor site after the injection of MGd (500 μM, 200 μL) into the tail vein of the mouse

FIG. 22 shows the normalized PA intensity changes over time (0-48 h) at the tumor site after the injection of ICG (500 μM, 200 mL; 5 μmol/kg) into the tail vein of the mouse. Pre=prior to injection of ICG.

FIG. 23 shows the PA and ultrasound overlaid images showing changes in PA signal over time with ICG (250 μM, 50 μL, 0.625 μmol/kg) and MMn (250 μM, 50 μL, 0.625 μmol/kg) being injected separately and directly at the tumor site. Laser light was used to illuminate the tumor during data collection: 800 nm, 20 mJ for ICG and 725 nm, 20 mJ for MMn.

FIG. 24 shows the normalized PA intensity changes over time (0-60 min) at the tumor site after the direct injection of ICG (250 μM, 50 μL, 0.625 μmol/kg) into the tumor of the mouse. Laser wavelength: 800 nm, Laser fluence: 20 mJ/cm². Percentage of PA signal after 1 hour=46%

FIG. 25 shows the normalized PA intensity changes over time (0-60 min) at the tumor site after the direct injection of MMn (250 μM, 50 μL; 0.625 μmol/kg) into the tumor of the mouse. Laser wavelength: 725 nm, Laser fluence: 20 mJ/cm². Percentage of PA signal after 1 hour=91%

FIG. 26 shows the 3D PA image of tumor after mice treated with MMn after 24 h. Excitation wavelength=725 nm. Laser fluence ˜18 mJ/cm².

FIG. 27 shows the representative Hematoxylin and Eosin (H&E) staining images of the major organs including the heart, liver, spleen, kidneys and lungs collected from the mice sacrificed 6 days post injection of MMn, MLu and MGd (Magnification: ×200)

FIGS. 28A-28D show the complete blood count tests of mice treated with PBS (control), MMn (500 μM, 200 μL; 5 μmol/kg), MLu (500 μM, 200 μL; 5 μmol/kg) and MGd (500 μM, 200 μL; 5 μmol/kg). (a) WBC: white blood cells; (b) RBC: red blood cells; (c) HGB: hemoglobin; (d) PLT: platelets.

FIG. 29 shows the temperature increase of solutions of MGd or MMn after irradiation in an in vitro sample.

FIG. 30 shows the change in temperature in PBS solutions of MGd, MLu, and MMn, at two different irradiation powers. The top images were irradiated at 3 W/cm² while the bottom images were irradiated at 6 W/cm².

FIG. 31 shows the viability of MDA-MB-231 cells after treating with MGd, MLu, and MMn.

FIGS. 32A & 32B show the photothermal imaging (FIG. 32A) of MGd, MLu, and MMn after irradiation with a 6 W light source at 808 nm in vitro. The graph (FIG. 32B) shows the viability of the cells after irradiation showing that both the MLu and MMn decreased viability after irradiation.

FIG. 33 shows the photothermal imaging of mice injected with MGd, MLu, and MMn.

FIG. 34 shows the temperature achieved using MMn, Mono-MMn, and Bis-MMn at various concentrations. On the right, the viability of the cells with and without light is shown in the graphs.

FIG. 35 shows the temperature achieved at various concentrations in PBS solution with a 2 W light source after 5 minute irradiation.

FIG. 36 shows the temperature achieved with 0-40 μM concentrations of MMn, Mono-MMn, and Bis-MMn along with the viability of the cells.

FIG. 37 shows the generation of cellular ROS after treatment with Bis-MMn.

FIG. 38 shows the photothermal effects of Bis-MMn after encapsulated in a liposome with increasing liposome amounts relative to the Bis-MMn.

FIG. 39 shows the in vivo photothermal effect of Bis-MMn encapsulated in a liposome recorded each minute for 5 minutes.

FIG. 40 shows the photothermal effect of liposomal encapsulated MGd, MLu, and MMn in PBS at 20 and 40 μM.

FIG. 41 shows the photothermal imaging of liposomal encapsulated MMn in vitro along with the related cell viability.

FIGS. 42A & 42B show the cell viability at 20 μM (FIG. 42A) and 40 μM (FIG. 42B).

### DESCRIPTION OF ILLUSTRATIVE EMBODIMENTS

The present disclosure relates to Mn containing texaphyrin compounds which may be used to image a patient either through photoacoustic imaging or MRI or used to treat the patient using photothermal therapy or delivery a therapeutic compound to a target tissue in the patient. These compounds may have one or more benefits. For example, the compounds may show more favorable photoacoustic imaging properties such as lower photobleaching or greater optical absorbance. Furthermore, the compounds that are described herein may also produce few side effects or generate fewer by-products such as singlet oxygen. Finally, the present compounds may have a more favorable toxicity profile than those known in the art. These and are more benefits of the claimed compounds are described herein.

### A. CHEMICAL DEFINITIONS

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means=NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “=” means a double bond, and “≡” means triple bond. The symbol “---” represents an optional bond, which if present is either single or double. The symbol “ [symbol] ” represents a single bond or a double bond. Thus, the formula

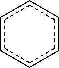

covers, for example,

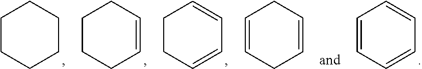

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

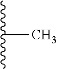

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

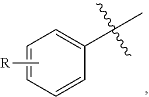

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

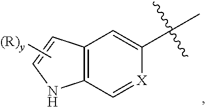

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

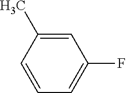

is also taken to refer to

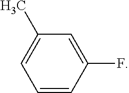

Aromatic compounds may also be depicted using a circle to represent the delocalized nature of the electrons in the fully conjugated cyclic π system, two non-limiting examples of which are shown below:

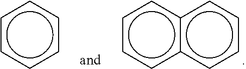

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

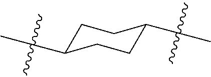 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

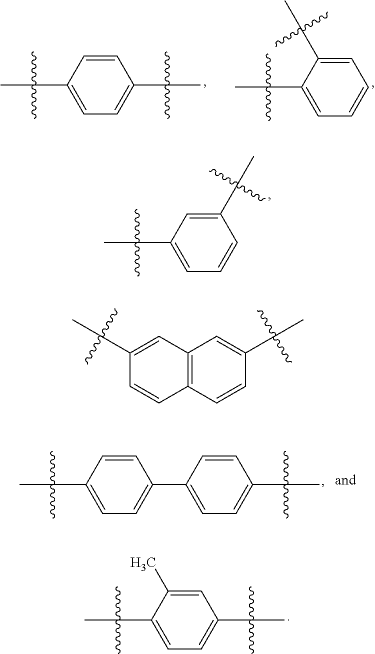

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. The term “heteroarenediyl” refers to a divalent aromatic group, with two aromatic carbon atoms, two aromatic nitrogen atoms, or one aromatic carbon atom and one aromatic nitrogen atom as the two points of attachment, said atoms forming part of one or more aromatic ring structures, each with three to eight ring atoms, wherein at least one of the ring atoms of the aromatic ring structure(s) is nitrogen, oxygen or sulfur, and wherein the divalent group consists of no atoms other than carbon, hydrogen, aromatic nitrogen, aromatic oxygen and aromatic sulfur. If more than one ring is present, the rings are fused; however, the term heteroarenediyl does not preclude the presence of one or more alkyl or aryl groups (carbon number limitation permitting) attached to one or more ring atoms. Non-limiting examples of heteroarenediyl groups include:

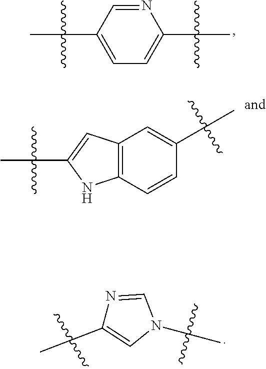 [SMILES: *c1ccc(*)nc1 ; *c1ccc2[nH]c(*)cc2c1 ; *c1cn(*)cn1]

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” refers to a divalent cyclic group, with two carbon atoms, two nitrogen atoms, or one carbon atom and one nitrogen atom as the two points of attachment, said atoms forming part of one or more ring structure(s) wherein at least one of the ring atoms of the non-aromatic ring structure(s) is nitrogen, oxygen or sulfur, and wherein the divalent group consists of no atoms other than carbon, hydrogen, nitrogen, oxygen and sulfur. If more than one ring is present, the rings are fused. As used herein, the term heterocycloalkanediyl does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkanediyl groups include:

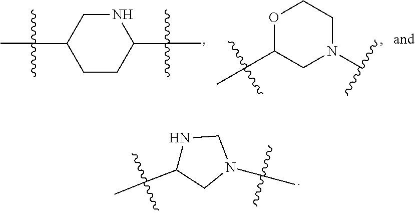 [SMILES: *C1CCC(*)NC1 ; *C1CN(*)CCO1 ; *C1CN(*)CN1]

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃. The terms “cycloalkylamino”, “alkenylamino”, “alkynylamino”, “arylamino”, “aralkylamino”, “heteroarylamino”, “heterocycloalkylamino”, and “alkoxyamino” when used without the “substituted” modifier, refers to groups, defined as —NHR, in which R is cycloalkyl, alkenyl, alkynyl, aryl, aralkyl, heteroaryl, heterocycloalkyl, and alkoxy, respectively. A non-limiting example of an arylamino group is —NHC₆H₅. The terms “dicycloalkylamino”, “dialkenylamino”, “dialkynylamino”, “diarylamino”, “diaralkylamino”, “diheteroarylamino”, “diheterocycloalkylamino”, and “dialkoxyamino”, refers to groups, defined as —NRR′, in which R and R′ are both cycloalkyl, alkenyl, alkynyl, aryl, aralkyl, heteroaryl, heterocycloalkyl, and alkoxy, respectively. Similarly, the term alkyl(cycloalkyl)amino refers to a group defined as —NRR′, in which R is alkyl and R′ is cycloalkyl. The amine versions of these compounds are compounds represent the compounds as noted above wherein the group is defined as: NNRR′. The term “alkylaminodiyl” when used without the “substituted” modifier refers to a divalent unsaturated aliphatic group, with zero, one, or two carbon atoms as points of attachment with the remaining points of attachment being nitrogen atoms, a linear or branched, a linear or branched acyclic structure containing at least one nitrogen atom in the chain, no nonaromatic carbon-carbon double bond, no carbon-carbon triple bonds, and no atoms other than carbon, nitrogen and hydrogen. The term alkylaminodiyl does not preclude the attachment of one or more additional alkyl groups on the nitrogen atoms to form tertiary amines carbon limit permitting.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

As used herein, the term “ligand” references to a chemical group which coordinates to a metal center through a bond. The bond between the ligand and the metal center in some cases is either an ionic or a coordination bond. A ligand can be monovalent, divalent, trivalent or have a greater valency. In some cases, a ligand may be negatively charged. Some exemplary examples of ligands include, but are not limited to, halide (F⁻, Cl⁻, Br⁻, or I⁻), a carbonate (CO₃²⁻), bicarbonate (HCO₃⁻), hydroxide (⁻OH), perchlorate (ClO₄⁻), nitrate (NO₃⁻), sulfate (SO₄²⁻), acetate (CH₃CO₂⁻), trifluoroacetate (CF₃CO₂⁻), acetylacetonate (CH₃COCHCOCH₃⁻), trifluorosulfonate (CF₃SO₂⁻), or phosphate (PO₄³⁻). A ligand could also be a neutral species that contains a lone pair of electrons. Some examples of neutral ligands include but are not limited to aqua (H₂O) or ammonia (NH₃). Additionally, a neutral ligand can include groups such as an alkylamine or a dialkylamine.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc.

In the context of this application, “selectively” means that greater than 50% of the activity of the compound is exhibited in the noted location. On the other hand, “preferentially” means that greater than 75% of the activity of the compound is exhibited in the noted location.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that for tetrahedral stereogenic centers the stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### B. COMPOUNDS OF THE PRESENT DISCLOSURE

The compounds of the present disclosure are shown, for example, above, in the summary of the invention section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present disclosure may contain one or more asymmetrically substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration. In some embodiments, the present compounds may contain two or more atoms which have a defined stereochemical orientation.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present disclosure function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### C. TEXAPHYRIN COMPOUNDS

In some aspects, the present disclosure provides compositions and methods comprising a texaphyrin compound with a platinum chelating group directly bound to the macrocycle, wherein the texaphyrin is a macrocycle of the formula:

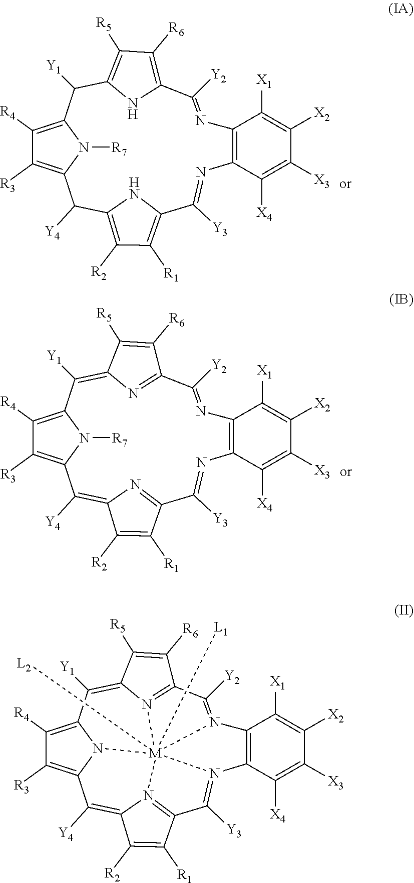

wherein:
Y₁-Y₄ are each independently selected from: hydrogen, amino, cyano, halo, hydroxy, or hydroxyamino,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤12)}, aryloxy_{(C≤12)}, heteroaryloxy_{(C≤12)}, heterocycloalkoxy_{(C≤12)}, amido_{(C≤12)}, alkylamino_{(C≤12)}, dialkylamino_{(C≤12)}, alkylthio_{(C≤12)}, arylthio_{(C≤12)}, alkylsulfinyl_{(C≤12)}, arylsulfinyl_{(C≤12)}, alkylsulfonyl_{(C≤12)}, arylsulfonyl_{(C≤12)}, or a substituted version of any of these groups; or
R₁-R₆ are each independently selected from: hydrogen, amino, cyano, halo, hydroxy, hydroxyamino, or nitro,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤12)}, aryloxy_{(C≤12)}, heteroaryloxy_{(C≤12)}, heterocycloalkoxy_{(C≤12)}, amido_{(C≤12)}, alkylamino_{(C≤12)}, dialkylamino_{(C≤12)}, or a substituted version of any of these groups; or
a PEG moiety wherein the PEG moiety is of the formula: —(OCH₂CH₂)ₙOR₈; wherein:
   n is 1-20; and
   R₈ is hydrogen, alkyl_{(C≤8)}, or substituted alkyl_{(C≤8)}; or
a platinum(IV) chelating group;
R₇ is hydrogen,
alkyl_{(C≤8)}, cycloalkyl_{(C≤8)}, alkenyl_{(C≤8)}, cycloalkenyl_{(C≤8)}, alkynyl_{(C≤8)}, alkoxy_{(C≤8)}, or a substituted version of any of these groups, or
an amino protecting group;
X₁-X₄ are each independently selected from: hydrogen, amino, cyano, halo, hydroxy, hydroxyamino, or nitro,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤12)}, aryloxy_{(C≤12)}, heteroaryloxy_{(C≤12)}, heterocycloalkoxy_{(C≤12)}, amido_{(C≤12)}, alkylamino_{(C≤12)}, dialkylamino_{(C≤12)}, or a substituted version of any of these groups; or
a PEG moiety wherein the PEG moiety is of the formula: —(OCH₂CH₂)ₙOR₈; wherein:
   n is 1-20; and
   R₈ is hydrogen, alkyl_{(C≤8)}, or substituted alkyl_{(C≤8)}; or
a platinum(IV) chelating group;
L₁ and L₂ are each independently absent, a neutral ligand, or an anionic ligand; and
M is a manganese ion;
provided that L₁ and L₂ are present or absent in a manner sufficient to balance the charge on the metal ion;
or a pharmaceutically acceptable salt or tautomer thereof.

In some embodiments, the platinum(IV) chelating group is further defined as:

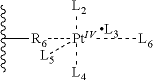

wherein:
   R₆ is carboxy;
   L₂-L₅ are each independently selected from ammonia, halide, alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
   L₆ is aqua, ammonia, nitrate, sulfate, halide, hydroxide, phosphate, or glucose-6-phosphate,
   alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups; and

Additional non-limiting examples of texaphyrins are taught by U.S. Pat. Nos. 4,935,498, 5,252,270, 5,272,142, 5,292,414, 5,369,101, 5,432,171, 5,439,570, 5,504,205, 5,569,759, 5,583,220, 5,587,463, 5,591,422, 5,633,354, 5,776,925, 5,955,586, 5,994,535, 6,207,660, 7,112,671, 8,410,263, and 10,406,167 which are all incorporated herein by reference. When the term, “texaphyrin compound” is used herein it can refer to a texaphyrin in both its oxidized and reduced forms, a metallotexaphyrin, or any of these groups. As would be known by a person of skill in the art, texaphyrins are known to undergo oxidation upon complexation of a metal ion. This phenomenon is described in U.S. Pat. No. 5,504,205, Shimanovich, et al., 2001 and Hannah, et al., 2001, all of which are incorporated herein by reference. As this process is linked with the metalation of the texaphyrin compound, these compounds are referenced to herein as an oxidized metallated derivative of the reduced macrocycle formula.

In some aspects, the present disclosure provides compositions and methods of use of the metallated form of the texaphyrin compound. In some embodiments, the metal of the metallated form is a transition metal. In some embodiments, the metal is a metal ion in the 2+ oxidation state or the 3+ oxidation state. The compounds described herein may contain a manganese atom in the complex. In some aspects, the texaphyrin compound is administered simultaneously with a reducing agent. In some embodiments, the reducing agent is a two electron donor. In some embodiments, the reducing agent is sodium ascorbate, thioredoxin reductase, a platinum(II) ion or complex, or a biological thiol, including but not limited to cysteine, homocysteine, or glutathione. A photoreduction may also be used in conjunction with the texaphyrin compound.

### D. HYPERPROLIFERATIVE DISEASES

While hyperproliferative diseases can be associated with any medical disorder that causes a cell to begin to reproduce uncontrollably, the prototypical example is cancer. One of the key elements of cancer is that the normal apoptotic cycle of the cell is interrupted and thus agents that lead to apoptosis of the cell are important therapeutic agents for treating these diseases. As such, the Mn texaphyrin analogues described in this disclosure may be effective in treating cancers.

Cancer cells that may be treated with the compounds according to the embodiments include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### E. PHOTOACOUSTIC IMAGING

Photoacoustic imaging (PAI) is an emerging biomedical imaging modality based on the photoacoustic effect. In PAI, light pulses (often from a laser) are delivered to a target locus (“situs”) in or on a sample. Some of the pulse energy is absorbed at the situs and converted into heat. The transient heating causes a corresponding transient thermoelastic expansion of the situs, which produces a corresponding wideband ultrasonic emission from the situs. The generated ultrasonic waves are detected using one or more ultrasonic transducers that convert the detected waves into corresponding electrical pulses that are processed into corresponding images. The optical absorption of light by a biological sample is closely associated with certain physiological properties such as hemoglobin concentration and/or oxygen saturation. As a result, the magnitude of ultrasonic emission (the photoacoustic signal, which is proportional to the local energy deposition) from the situs reveals physiologically specific optical absorption contrast that facilitate formation of 2-D or 3-Dl images of the situs. Blood usually exhibits greater absorption than surrounding tissues, which provides sufficient endogenous contrast to allow PAI of blood vessels and tissues containing same. For example, PAI can produce high-contrast images of breast tumors in situ due to the greater optical absorption by the increased blood supply provided by the body to the tumor. Whereas conventional X-ray mammography and ultrasonography produce images of benign features as well as pathological features, PAI can produce information more specific to the malignant condition, such as enhanced angiogenesis at the tumor site.

Significant challenges currently limit PAI from widespread clinical use including both in the imaging systems as well as contrast agents. The development of contrast agents has been carried out in order to assist in generating images using PAI. Examples of contrast agents include organic based contrast agents such as cyanine dyes, nanoparticles, polyhydroxy-fullerene and carbon nanotubes. However, the photoacoustic contrast that is achieved using the contrast agents has often been low, resulting in poor spatial resolution of the images. Therefore, the development of new PAI contrast agents could result in useful in making PAI clinically useful.

### F. PHOTOTHERMAL THERAPY

Additionally, photothermal therapy is a technique for treating cancer or related diseases by transferring a substance (photothermal material) capable of absorbing light to generate heat to a lesion site and then irradiating light to generate heat. This photothermal therapy is a method of selectively killing only cancer cells that relies on the fact that cancer cells are typically less resistant to heat than normal cells. Photothermal therapy is currently one of the more extensively researched technologies in the field of cancer treatment. Near-infrared rays are used as light used for photothermal treatment, since the encompass wavelengths that are relatively harmless to normal cells.

However, one of challenges associated with photothermal treatment is that the photothermal material administered in vivo to absorb light and generate beat may not be cleared out of the body in a timely manner. The field of photothermal treatment often makes use of metal nanoparticles to absorb light and generate heat. However, such inorganic materials are often not smoothly discharged when administered in vivo, potentially posing safety concerns. Therefore, compounds that may be used in photothermal treatments which have improved safety profiles would address an unmet need.

### Pharmaceutical Formulations and Routes of Administration

For administration to a mammal in need of such treatment, the Mn texaphyrin analogues of the present disclosure are ordinarily combined with one or more excipients appropriate to the indicated route of administration. The Mn texaphyrin analogues of the present disclosure may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol, and tableted or encapsulated for convenient administration. Alternatively, the Mn texaphyrin analogues of the present disclosure may be dissolved in water, polyethylene glycol, propylene glycol, ethanol, corn oil, cottonseed oil, peanut oil, sesame oil, benzyl alcohol, sodium chloride, and/or various buffers. Other excipients and modes of administration are well and widely known in the pharmaceutical art.

The pharmaceutical compositions useful in the present disclosure may be subjected to conventional pharmaceutical operations such as sterilization and/or may contain conventional pharmaceutical carriers and excipients such as preservatives, stabilizers, wetting agents, emulsifiers, buffers, etc.

The Mn texaphyrin analogues of the present disclosure may be administered by a variety of methods, e.g., orally or by injection (e.g. subcutaneous, intravenous, intraperitoneal, etc.). Depending on the route of administration, the novel conjugates may be coated in a material to protect the compound from the action of acids and other natural conditions which may inactivate the compound. They may also be administered by continuous perfusion/infusion of a disease or wound site.

To administer the therapeutic compound by other than parenteral administration, it may be necessary to coat the Mn texaphyrin analogues of the present disclosure with or co-administer the Mn texaphyrin analogues of the present disclosure with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes. Additionally, Trapasol®, Travasol®, cyclodextrin, and other drug carrier molecules may also be used in combination with the Mn texaphyrin analogues of the present disclosure. It is contemplated that the compounds of the present disclosure may be formulated with a cyclodextrin as a drug carrier using an organic solvent such as dimethylaceamide with a polyethylene glycol and a poloxamer composition in an aqueous sugar solution. In some embodiments, the organic solvent is dimethylsulfoxide, dimethylformamide, dimethylacetamide, or other biologically compatible organic solvents. Additionally, the composition may be diluted with a polyethylene glycol polymer such as PEG100, PEG200, PEG250, PEG400, PEG500, PEG600, PEG750, PEG800, PEG900, PEG1000, PEG2000, PEG2500, PEG3000, or PEG4000. Additionally, the composition may further comprise one or more poloxamer composition wherein the poloxamer contains two hydrophilic polyoxyethylene groups and a hydrophobic polyoxypropylene or a substituted version of these groups. This mixture may be further diluted using an aqueous sugar solution such as 5% aqueous dextrose solution.

The Mn texaphyrin analogues of the present disclosure may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion are also envisioned. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating Mn texaphyrin analogues of the present disclosure in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The Mn texaphyrin analogues of the present disclosure can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the Mn texaphyrin analogues of the present disclosure in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of the Mn texaphyrin analogues of the present disclosure calculated to produce the desired therapeutic effect in association with the required pharmaceutical carrier. The specification for the dosage unit forms of the invention are dictated by and directly dependent on (a) the unique characteristics of the Mn texaphyrin analogues of the present disclosure and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation.

The Mn texaphyrin analogues of the present disclosure describe in this disclosure are administered at a therapeutically effective dosage sufficient to treat a condition associated with a condition in a patient. For example, the efficacy of the Mn texaphyrin analogues of the present disclosure can be evaluated in an animal model system that may be predictive of efficacy in treating the disease in humans, such as the model systems shown in the examples and drawings.

The actual dosage amount of the Mn texaphyrin analogues of the present disclosure comprising the compounds of the present disclosure administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

HED(mg/kg)=Animal dose(mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

An effective amount typically will vary from about 1 mg/kg to about 50 mg/kg, in one or more dose administrations daily, for one or several days (depending of course of the mode of administration and the factors discussed above). In some particular embodiments, the amount is less than 5,000 mg per day with a range of 10 mg to 4500 mg per day.

The effective amount may be less than 10 mg/kg/day, less than 50 mg/kg/day, less than 100 mg/kg/day, less than 250 mg/kg/day. It may alternatively be in the range of 1 mg/kg/day to 250 mg/kg/day.

In other non-limiting examples, a dose may also comprise from about 0.1 mg/kg/body weight, about 1 mg/kg/body weight, about 10 g/kg/body weight, about 50 g/kg/body weight, or more per administration, and any range derivable therein. In non-limiting examples of a derivable range from the numbers listed herein, a range of about 1 mg/kg/body weight to about 50 mg/kg/body weight, about 5 g/kg/body weight to about 10 g/kg/body weight, etc., can be administered, based on the numbers described above.

In certain embodiments, a pharmaceutical composition of the present disclosure may comprise, for example, at least about 0.1% of a compound described in the present disclosure. In other embodiments, the compound of the present disclosure may comprise between about 0.25% to about 75% of the weight of the unit, or between about 25% to about 60%, or between about 1% to about 10%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agents are administered once a day.

The compounds may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may take orally and that the timing of which is or is not dependent upon food intake. Thus, for example, the agents can be taken every morning and/or every evening, regardless of when the subject has eaten or will eat.

### G. COMBINATION THERAPY

In addition to being used as a monotherapy, the Mn texaphyrin analogues of the present disclosure may also find use in combination therapies. Effective combination therapy may be achieved with a single composition or pharmacological formulation that includes both agents, or with two distinct compositions or formulations, administered at the same time, wherein one composition includes a Mn texaphyrin analogues and compositions, and the other includes the second agent(s). The other therapeutic modality may be administered before, concurrently with, or following administration of the Mn texaphyrin analogues of the present disclosure. The therapy using the Mn texaphyrin analogues of the present disclosure may precede or follow administration of the other agent(s) by intervals ranging from minutes to weeks. In embodiments where the other agent and the compounds or compositions of the present disclosure are administered separately, one would generally ensure that a significant period of time did not expire between the time of each delivery, such that each agent would still be able to exert an advantageously combined effect. In such instances, it is contemplated that one would typically administer the Mn texaphyrin analogues of the present disclosure and the other therapeutic agent within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of a Mn texaphyrin analogues of the present disclosure or the other agent will be desired. In this regard, various combinations may be employed. By way of illustration, where the compounds of the present disclosure are “A” and the other agent is “B”, the following permutations based on 3 and 4 total administrations are exemplary:
A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated. Non-limiting examples of pharmacological agents that may be used in the present invention include any pharmacological agent known to be of benefit in the treatment of a cancer or hyperproliferative disorder or disease. In some embodiments, combinations of the Mn texaphyrin analogues of the present disclosure with a cancer targeting immunotherapy, radiotherapy, chemotherapy, or surgery are contemplated. Also contemplated is a combination of the Mn texaphyrin analogues of the present disclosure with more than one of the above-mentioned methods including more than one type of a specific therapy. In some embodiments, it is contemplated that the immunotherapy is a monoclonal antibody which targets HER2/neu such trastuzumab (Herceptin®), alemtuzumab (Sampath®), bevacizumab (Avastin®), cetuximab (Eribitux®), and panitumumab (Vectibix®) or conjugated antibodies such as ibritumomab tiuxetan (Zevalin®), tositumomab (Bexxar®), brentuximab vedotin (Adcetris®), ado-trastuzumab emtansine (Kadcyla™), or denileukin dititox (Ontak®) as well as immune cell targeting antibodies such as ipilimumab (Yervoy®), tremelimumab, anti-PD-1, anti-4-1-BB, anti-GITR, anti-TIM3, anti-LAG-3, anti-TIGIT, anti-CTLA-4, or anti-LIGHT. Furthermore, in some embodiments, the texaphyrin-platinum(IV) conjugate the composition of a Mn texaphyrin analogues of the present disclosure are envisioned to be used in combination therapies with dendritic cell-based immunotherapies such as Sipuleucel-T (Provenge®) or adoptive T-cell immunotherapies.

Furthermore, it is contemplated that the methods described herein may be used in combination with a chemotherapeutic agent such as PR-171 (Kyprolis®), bortezomib (Velcade®), anthracyclines, taxanes, methotrexate, mitoxantrone, estramustine, doxorubicin, etoposide, vinblastine, vinorelbine, 5-fluorouracil, cisplatin, carboplatin, oxaliplatin, Pt(IV) complexes, topotecan, ifosfamide, cyclophosphamide, epirubicin, gemcitabine, vinorelbine, irinotecan, etoposide, vinblastine, pemetrexed, melphalan, capecitabine, oxaliplatin, BRAF inhibitors, and TGF-beta inhibitors. In some embodiments, the combination therapy is designed to target a cancer such as those listed above.

In some aspects, it is contemplated that the Mn texaphyrin analogues of the present disclosure may be used in conjunction with radiation therapy. Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of y-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Additionally, it is contemplated Mn texaphyrin analogues of the present disclosure are used in combination with sonodynamic therapy. The use of texaphyrins in sonodynamic therapy is described in U.S. Pat. No. 6,207,660 incorporated herein by reference. A Mn texaphyrin analogues of the present disclosure is administered before administration of the sonodynamic agent. The compound may be administered as a single dose, or it may be administered as two or more doses separated by an interval of time. Parenteral administration is typical, including by intravenous and interarterial injection. Other common routes of administration can also be employed.

Ultrasound is generated by a focused array transducer driven by a power amplifier. The transducer can vary in diameter and spherical curvature to allow for variation of the focus of the ultrasonic output. Commercially available therapeutic ultrasound devices may be employed in the practice of the disclosure. The duration and wave frequency, including the type of wave employed may vary, and the preferred duration of treatment will vary from case to case within the judgment of the treating physician. Both progressive wave mode patterns and standing wave patterns have been successful in producing cavitation of diseased tissue. When using progressive waves, the second harmonic can advantageously be superimposed onto the fundamental wave.

Preferred sonodynamic agents employed in the present disclosure is ultrasound, particularly is low intensity, non-thermal ultrasound, i.e., ultrasound generated within the wavelengths of about 0.1 MHz and 5.0 MHz and at intensities between about 3.0 and 5.0 W/cm².

Furthermore, it is contemplated that the compounds of the present disclosure can be used in combination with photodynamic therapy: By way of example, a texaphyrin is administered in solution containing 2 mg/ml optionally in 5% mannitol, USP. Dosages of about 1.0 or 2.0 mg/kg to about 4.0 or 5.0 mg/kg, preferably 3.0 mg/kg may be employed, up to a maximum tolerated dose that was determined in one study to be 5.2 mg/kg. The texaphyrin is administered by intravenous injection, followed by a waiting period of from as short a time as several minutes or about 3 hours to as long as about 72 or 96 hours (depending on the treatment being effected) to facilitate intracellular uptake and clearance from the plasma and extracellular matrix prior to the administration of photoirradiation.

The co-administration of a sedative (e.g., benzodiazapenes) and narcotic analgesic are sometimes recommended prior to light treatment along with topical administration of Emla cream (lidocaine, 2.5% and prilocaine, 2.5%) under an occlusive dressing. Other intradermal, subcutaneous and topical anesthetics may also be employed as necessary to reduce discomfort. Subsequent treatments can be provided after approximately 21 days. The treating physician may choose to be particularly cautious in certain circumstances and advise that certain patients avoid bright light for about one week following treatment.

When employing photodynamic therapy, a target area is treated with light at about 732±16.5 nm (full width half max) delivered by LED device or an equivalent light source (e.g., a Quantum Device Qbeam™ Q BMEDXM-728 Solid State Lighting System, which operates at 728 nm) at an intensity of 75 mW/cm² for a total light dose of 150 J/cm². The light treatment takes approximately 30 minutes.

The optimum length of time following texaphyrin administration until light treatment can vary depending on the mode of administration, the form of administration, and the type of target tissue. Typically, the texaphyrin persists for a period of minutes to hours, depending on the texaphyrin, the formulation, the dose, the infusion rate, as well as the type of tissue and tissue size.

After the photosensitizing texaphyrin has been administered, the tissue being treated is photoirradiated at a wavelength similar to the absorbance of the texaphyrin, usually either about 400-500 nm or about 700-800 nm, more preferably about 450-500 nm or about 710-760 nm, or most preferably about 450-500 nm or about 725-740 nm. The light source may be a laser, a light-emitting diode, or filtered light from, for example, a xenon lamp; and the light may be administered topically, endoscopically, or interstitially (via, e.g., a fiber optic probe). Preferably, the light is administered using a slit-lamp delivery system. The fluence and irradiance during the photoirradiating treatment can vary depending on type of tissue, depth of target tissue, and the amount of overlying fluid or blood. For example, a total light energy of about 100 J/cm² can be delivered at a power of 200 mW to 250 mW depending upon the target tissue.

One aspect of the present invention is that the compounds of the present disclosure can additionally be used to image the localization of the therapeutic agent. The texaphyrin core allows for the use of MRI to determine the location of the compound with the patient and determine the specific location and margin of the tumor to which it has localized. In some aspects, the ability to determine the location of the texaphyrin core may be advantageous for more or additional therapeutic methods such as surgery or radiation therapy.

### H. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Synthesis of Compounds

### A. Materials and Methods

Starting materials were purchased from Fisher Scientific or Sigma Aldrich and used without further purification unless otherwise specified. Oxaliplatin (IV) prodrug (1) was synthesized according to our previously reported procedure (1). Indocyanine green (ICG) was purchased from by Dandong Yichuang Pharmaceutical Co., Ltd. Solvents were purified using a solvent purifier system (Vacuum Atmospheres). Analytical RP-HPLC analyses for MMn, MGd and MLu were performed on a Thermo scientific Dionex Ultimate 3000 equipped with a PDA detector. The analytical column was a Syncronis C18 column, 5 μm, 4.6×250 mm (Thermo Scientific); the mobile phase consisted of an increasing gradient (from 10% to 99% in 20 min) of acetonitrile into water, both containing 0.1% acetic acid. In this case the flow rate was 1.2 mL/min. Texaphyrins were monitored at 254, 470, and 740 nm. MMn, MGd, and MLu were purified on reverse phase-t C18 SPE (Waters Sep-Pak, Waters) columns containing 10 g of C-18 using an increasing gradient of acetonitrile in either 0.1 M ammonium acetate/1% acetic acid aqueous solution or 0.1 M potassium nitrate aqueous solution as the eluent, depending on which counter anion (AcO⁻ or NO₃⁻) was desired as the ancillary ligand. Mass spectrometric analyses were carried out in The University of Texas at Austin Mass Spectrometry Facility. Low-resolution and high-resolution electro-spray mass spectrometric (ESI-MS) analyses were carried out using Thermo Finnigan LTQ and Qq-FTICR (7 Telsa) instruments, respectively. UV-Vis spectra were recorded using a Varian Cary 5000 spectrophotometer at room temperature. The UV-Vis absorption and fluorescence emission spectra of the resulting solutions were measured by a Varian Cary 500 UV-Vis spectrophotometer and Varian Cary eclipse fluorescence spectrophotometer, respectively. A quartz cuvette with a cell length of 10 mm was used in all UV-Vis and fluorescence studies. The purity of the texaphyrin derivatives, MMn—NH₂, Mono-MMn, Bis-MMn, MMn, MLu, and MGd was checked by reverse phase-HPLC before use. For those known compounds, MMn, MLu, and MGd matched previously reported analysis (Brewster et al., 2020). LogP values and the protocol for their determination can be found in the previous report by Brewster et al (2020).

### B. Synthesis and Characterizations of MGd, MLu and MMn

MGd was prepared as described in Sessler et al., 1993. MLu and MMn were synthesized using literature protocols (Blumenkranz et al., 2000; Shimanovich et al., 2001; Sessler et al., 1993).
i.

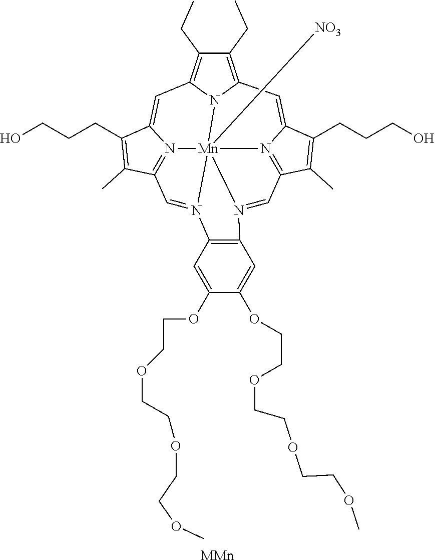

MMn was synthesized according to a previously reported procedure (Shimanovich et al., 2001). Upon reaction completion, MMn was purified via silica column chromatography (DCM/MeOH—100/0 to 95/5 then 80/20). Further purification was performed using reverse phase tC18 SPE (Waters Sep-Pak, Waters) columns containing 10 g of the C18 support using an increasing gradient of acetonitrile and 0.1% acetic acid aqueous solution. Final step of purification, Sep-Pak loaded MMn was washed with a 0.1 M potassium nitrate aqueous solution three times followed by a final wash of deionized H₂O. MMn was eluted using MeOH and the solvent was removed in vacuo to afford MMn as a crystalline green solid.
ii.

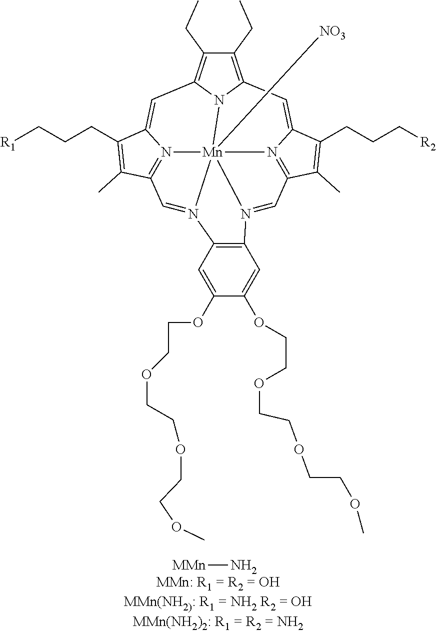

MMn(NH₂) and MMn(NH₂)₂ were synthesized following a previously published procedure for MGd(NH₂) derivatives but modified for MMn derivatives (Thiabaud et al., 2020). In brief, MMn and 3 equiv. of each of the reagents (triphenylphosphine, phthalimide and diisopropylazodicarboxylate) in DCM was used to form the corresponding pthalimide derivatives. Upon achieving the desired ratios of intermediates, the reaction was subjected to a short silica column (DCM/MeOH—100/0 to 95/5 then 80/20). Phthalimide deprotection using methylamine (40% in water) was used to achieve a mixture of mono-, bis-NH₂, and unreacted MMn. These three compounds were separated on reverse phase silica gel chromatography column using reverse phase tC18 SPE with an increasing gradient of acetonitrile and 0.1% acetic acid aqueous solution. Final step of purification, Sep-Pak loaded MMn derivative was washed with a 0.1 M potassium nitrate aqueous solution three times followed by a final wash of deionized H₂O. MMn derivative was eluted using MeOH and the solvent was removed in vacuo to afford the desired product as a crystalline green solid.
iii.

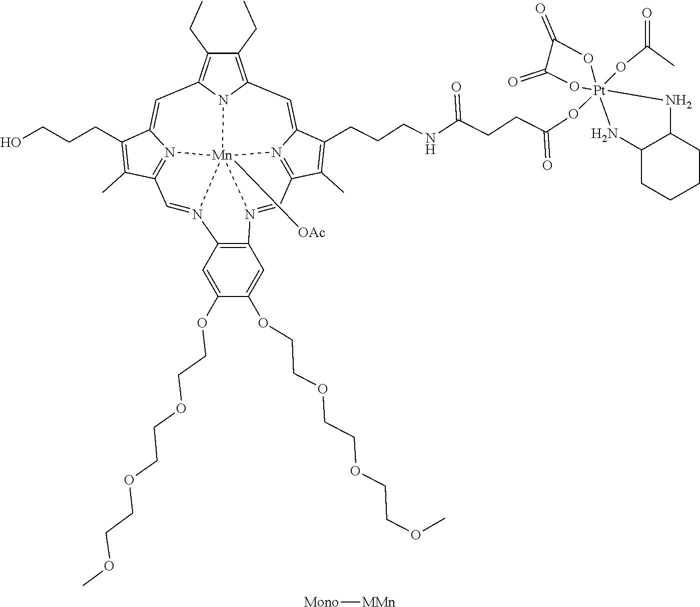

Mono-MMn was synthesized following a similar protocol published protocol (Thiabaud et al., 2014). In brief, 1.5 equiv. of NHS, 1 and EDC in H₂O was stirred for 20 minutes. Activated 1 was then added to a solution of 1 equiv. of DIPEA and MMn(NH₂) in MeCN and the reaction was stirred for approx. 6 hours. Mono-MMn was then purified via reverse phase tC18 SPE with an increasing gradient of acetonitrile and 0.1% acetic acid aqueous solution. Final step of purification, Sep-Pak loaded Mono-MMn was washed with deionized H₂O and eluted using MeOH and the solvent was removed in vacuo to afford the desired product as a crystalline green solid. High Resolution ESI-MS m/z 1482.5212 (MonoMMn—OAc)⁺iv.

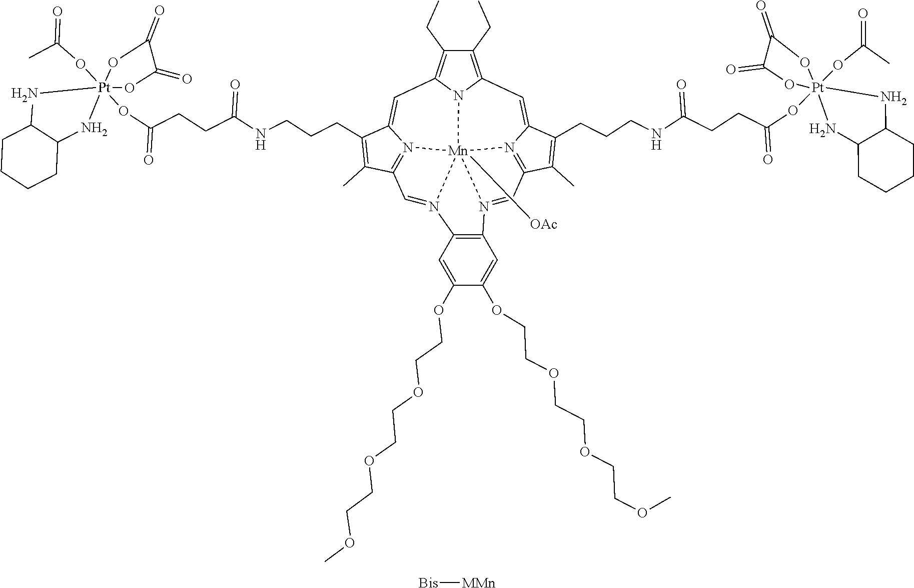

Bis-MMn was synthesized using similar protocol published protocol (Thiabaud et al., 2014). For the synthesis, 3 equiv. of NHS, 1 and EDC in H₂O was stirred for 20 minutes. Activated 1 was then added to a solution of 2 equiv. of DIPEA and lequiv. of MMn(NH₂)₂ in MeCN and the reaction was stirred for approx. 6 hours. Bis-MMn was then purified via reverse phase tC18 SPE with an increasing gradient of acetonitrile and 0.1% acetic acid aqueous solution. Final step of purification, Sep-Pak loaded Mono-MMn was washed with deionized H₂O and eluted using MeOH and the solvent was removed in vacuo to afford the desired product as a crystalline green solid. High Resolution ESI-MS m/z 1482.5212 (BisMMn—OAc)⁺

### Example 2: Photoacoustic Imaging with Manganese Compounds

### A. Methods and Materials

i. Cell Culture Imaging Protocol

RAW 264.7 cells were purchased from a commercial source and cultured in Dulbecco's Modified Eagle Medium (DMEM) (Life Technologies, NY, USA) supplemented with 10% fetal bovine serum (FBS; HyClone, UT, USA). Cells were incubated at 37° C. in a humidified atmosphere containing 5% CO₂. The cells were then divided into 4 groups and cultured in culturing bottle (75 mL); MMn, MLu and MGd were mixed in the cell media (no FBS) at a concentration of 500 μM and incubated with cells for 30 mins. The old media was then discarded, and the cells were washed with PBS three times and collected by centrifugation (2000 rpm, 2 mins; or 3000-5000 rpm, 1 min). The collected cells were then added into the plastic tube for PA measurements.

ii. In Vivo Imaging Protocol

Male BALB/c nude mice (18±2 g, 4-6 weeks old) were purchased from a commercial source and used according to standard animal guidelines for the care and use of laboratory animals with appropriate ethical oversight board approval.

Human prostate cancer cell lines C4-2 were purchased from commercial sources and cultured in Dulbecco's Modified Eagle Medium (DMEM) (Life Technologies, NY, USA) supplemented with 10% fetal bovine serum (FBS; Cyclone, UT, USA). Cells were incubated at 37° C. in a humidified atmosphere containing 5% CO₂. For C4-2 tumor inoculation, C4-2 cells (˜1×10⁶) suspended in PBS were injected under the nude mice epidermis of the right flank. Animals were observed every day for any behavioral abnormalities. Approximately after 12 days, the tumors were a suitable size for PAI experiments (diameter of tumor: ˜8-12 mm). Before each PAI experiment, the tumor site was gently wiped with alcohol and the tumor was first coated with an ultrasonic coupling adhesive and then a polyethylene plastic film, which held water was directly placed above the tumor. This protocol was important as the acoustic impedance mismatch between tissue and air would prevent the ultrasound information being received

iii. Photoacoustic Computed Tomography (PACT) System for In Vivo Experiments

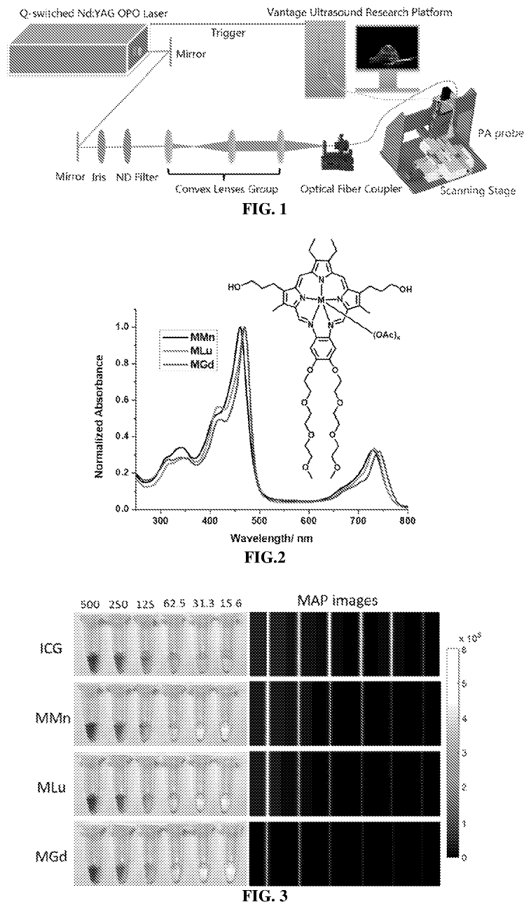

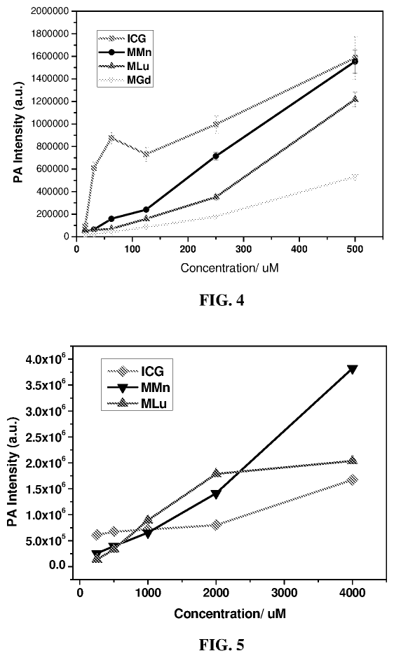

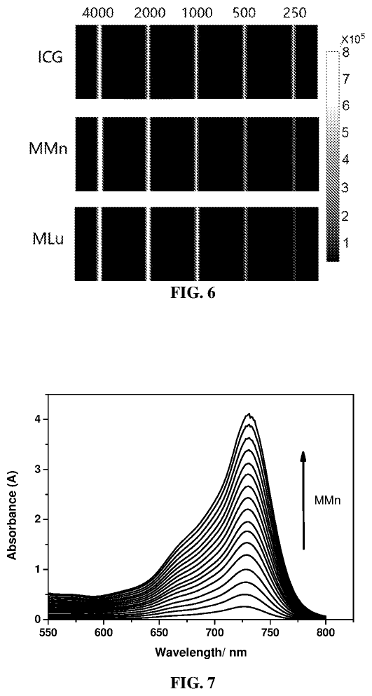

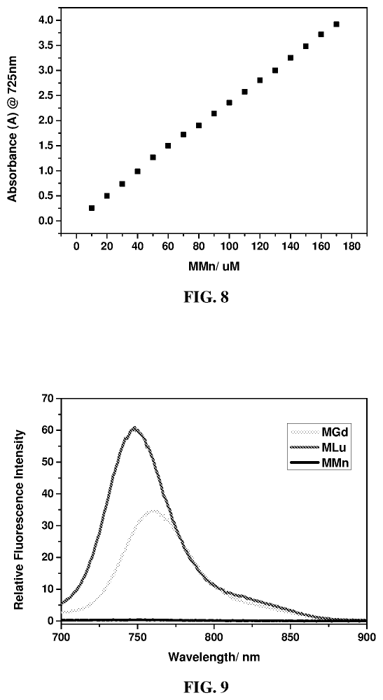

An optical parametric oscillator (OPO) laser source (Innolas GmbH, Bonn, Germany) emitting 8 ns pulsed lasers was coupled to a multimode optical fiber with a 1500 μm core diameter for the photoacoustic signal excitation. The use of this nanosecond pulsed laser on the tumor tissue resulted in very small thermal expansions. This led to the vibration of the tissue, which generated ultrasonic waves that could be recorded by a commercial 128-element linear array transrectal ultrasound transducer. For a model apparatus, see FIG. 1. The Q-switch output of the laser source was synchronized with a Vantage 128 research ultrasound platform (Verasonics, Inc. Kirkland WA, USA) to perform photoacoustic and ultrasound data acquisition. Photoacoustic signals were acquired at a frame rate equal to the laser pulse firing frequency of 30 Hz, and the conventional delay and sum (DAS) reconstruction algorithm was used to reconstruct the photoacoustic B-scan image (Harrison and Zemp, 2011). The system can simultaneously display both photoacoustic and ultrasound images in real-time. The following excitation wavelengths were used for each texaphyrin contrast agent: MMn: 725 nm and MGd: 741 nm

iv. ICP-MS Analysis Protocol

MMn or MGd were mixed in cell media (no FBS, 15 mL, Cytiva HyClone DMEM, SH300243.01) to achieve a concentration of 100 μM for each texaphyrin and incubated (37° C., 95% air, 5% CO₂) with RAW 264.7 cells for 30 mins in a 75 mL culture flask. The old media was discarded, and the cells were washed with PBS three times. The cells were detached using trypsin and then collected by centrifugation (2000 rpm, 2 min). The collected cells were then added into a centrifuge tube. Nitric acid (1 mL; 65%-68%) was then added to the tube dropwise which was then held in a water bath at 90° C. for 1 hour with the tube sealed. Upon cooling to room temperature, 300 μL H₂O₂ (30%) was added to the tube, which was unsealed and placed in a water bath at 90° C. for an additional 1 hour. The tubes were then cooled to room temperature and the contents filtered using a 0.45 um filter membrane. The filtrate was then subject to ICP-MS analysis (Thermo Scientific, iCAP™ Q).

### B. Photoacoustic Properties of Manganese Derivatives

The attributes of MGd such as attractive safety profile, MRI enhancement, (Young et al., 1996) (Sedgwick et al., 2020) and tumor localization (Hashemy et al., 2007; Khuntia 2007), coupled with a strong absorptivity in the near-IR region >700 nm (FIG. 2) led us to test metallotexaphyrins as potential PA contrast agents. These compounds when tested for their photoacoustic properties were found to be potential PA contrast agents. In particular, MMn, (Shimanovich et al., 2001) the manganese(II) analogue of MGd and MLu, provides a superior and effective PA contrast agent with greater photostability than ICG. Given this recently discovered PA contrast agent, MMn is particularly appealing in that the compound has demonstrated MRI enhancing capabilities (Brewster et al., 2020; Keca et al., 2016) and the low toxicity profile of the texaphyrin compounds (Young et al., 1996) (Sedgwick et al., 2020; Hashemy et al., 2007; Khuntia 2007). For the photoacoustic imaging (PAI) experiments, each compound was dissolved in doubly distilled water and then each solution was placed into a 1.1 mm diameter plastic tube and subjected to a laser fluence of 5 mJ/cm². The maximum amplitude projection (MAP) image was then captured (FIG. 3). Under these test conditions, ICG demonstrated the greatest PA intensities across the concentration range chosen for initial study (15.6-500 μM). However, MMn displayed the greatest PA intensities in comparison to MGd and MLu, with MGd being the least effective (FIGS. 3 & 4). Additionally, at the relatively high concentrations (>1 mM) that might be needed for MRI studies, MMn displayed a PA intensity that was greater than that of ICG (FIGS. 5 & 6). Without wishing to be bound by any theory, it is believed to be the result of the aggregation of ICG resulting in the reduction of the UV-Vis absorption and PA intensity at 800 nm, (Weigand et al., 1997; Penzkofer et al., 1996). No evidence of aggregation was seen for MMn (FIGS. 7 & 8).

As PAI utilizes the nonradiative conversion of light energy, (Liu et al., 2019) a desired feature of a PA contrast agent is a low fluorescent quantum yield (Borg et al., 2018). Therefore, without wishing to be bound by any theory, it is believed that the superior performance observed for MMn comparison to MGd and MLu reflects that the compound has being essentially non-fluorescent (FIG. 9). This difference in photophysical properties is the result of the texaphyrin ligand chromophore being strongly influenced by the complexed metal cation (Guldi et al., 2000) Mn(II) is a well-known fluorescence quencher, (Hannah et al., 2002; Volchkov et al., 2010; Choi and Luo 2018; Senthilnithy et al. 2009) that promotes efficient non-radiative decay and effective PA imaging in the case of MMn.

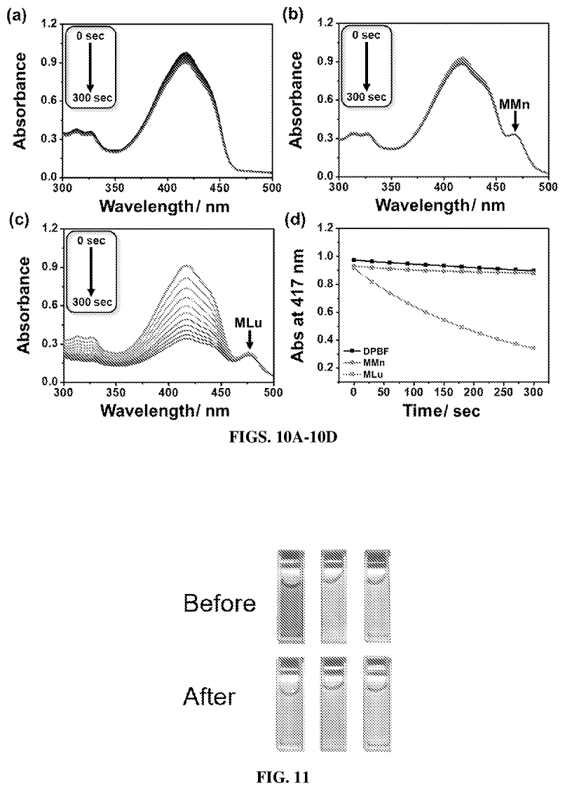

A preferred PA contrast agent for clinical applications should avoid production of singlet oxygen (¹O₂). Surprisingly, MMn resulted in minimal production of ¹O₂, as reflected in a standard 1,3-diphenylisobenzofuran (DPBF)-based assay (FIG. 10) (Seto et al., 2016). In contrast, photoirradiation of MLu resulted in the expected production of ¹O₂. Due to its ¹O₂ sensitizing properties, MLu was deemed less attractive then MMn as a potential PA contrast agent.

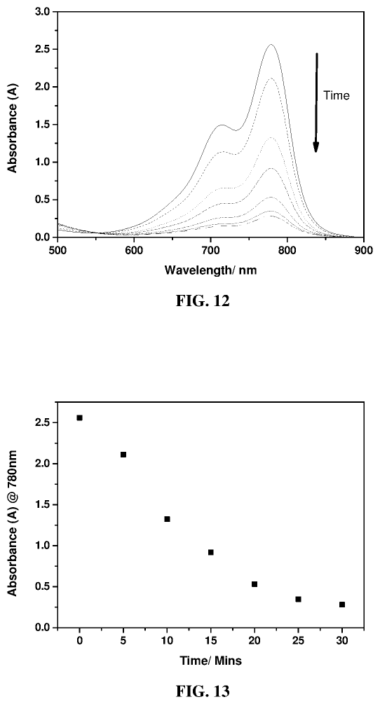

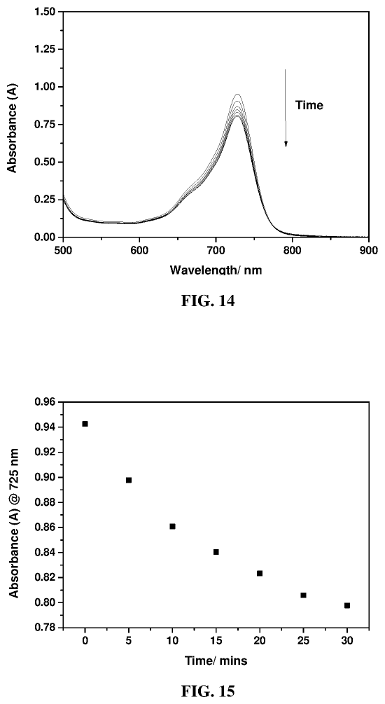

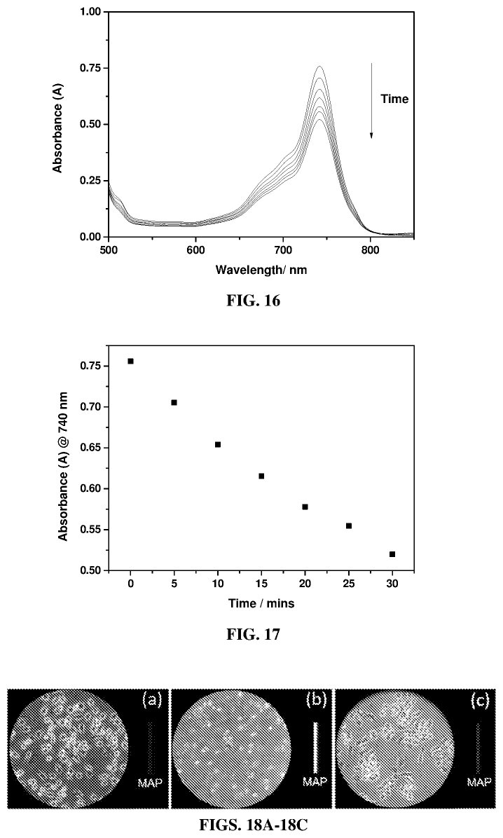

Photostability is also relevant factor to consider for a potential PA contrast agent for clinical applications. Consistent with previous observations, (Mindt et al., 2018) ICG demonstrated poor photostability upon photoirradiation (100 μM, 20 minutes, 780 nm, fluence ˜18 mJ/cm²) as inferred from the change in color from dark turquoise to a pale turquoise (cf. FIG. 11). A plot of intensity at 780 nm vs time for ICG demonstrated an overall 90% change in absorption over 30 min photoirradiation (40 μM, 30 minutes, 780 nm, fluence ˜18 mJ/cm²), indicative of significant photodegradation (FIGS. 12 & 13). A corresponding analysis revealed MMn as possessing a greater photostability than either ICG or MGd (40 μM, 20 minutes, 725 nm (MMn) and 740 nm (MGd); fluence ˜18 mJ/cm²) as reflected in a 15% and 30% change in absorbance intensity for MMn and MGd, respectively (FIGS. 14-17).

The intracellular PAI capabilities of MMn and MGd were evaluated in RAW 264.7 cells, a cell line commonly used in imaging assays (Sedgwick et al., 2018). As can be seen from an inspection of FIG. 18, MMn when incubated at a concentration of 500 μM produced a strong intracellular PA signal (PA intensity=6.75×10⁶). In contrast, a lower PA intensity was observed for MGd (PA intensity=3.63×10⁵) and the control (doubly distilled water; intensity=1.71×10⁵). ICP-MS analysis revealed a 2.4-fold greater Mn content in RAW 264.7 cells treated with MMn than those treated with MGd under otherwise identical conditions (Table 1). The enhanced intracellular PA intensity seen for MMn is believed to be due to its greater cellular uptake combined with its favorable PA properties.

TABLE 1
ICP-MS analysis of the cellular uptake
of either Mn or Gd in RAW264.7 cells

| | Concentration | | Concentration | Intensity |
|---|---|---|---|---|
| Category | average | | RSD | average |
| ⁵⁵Mn (KED) | 220.413 | ppb | 0.5% | 1,566,566 cps |
| ¹⁵⁷Gd (KED) | 91.783 | ppb | 0.5% | 2,823,850 cps |

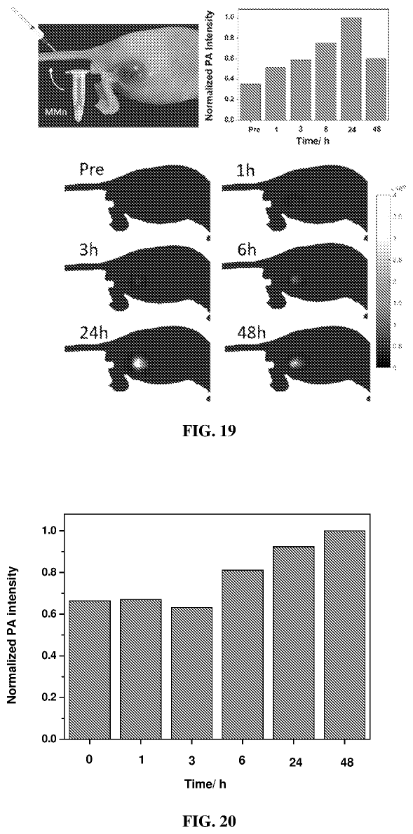

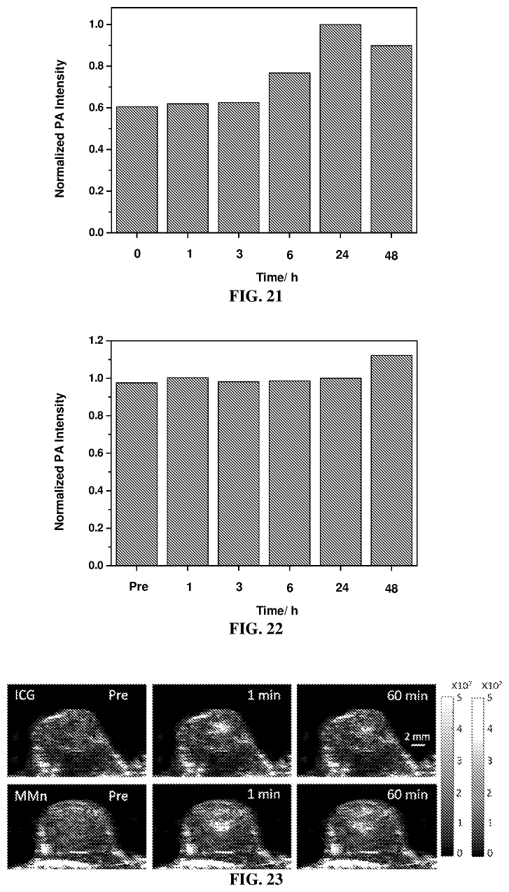

To test further the PA imaging potential of MMn, in vivo experiments were performed using a prostate tumor mouse model as shown in FIG. 19. The tumors were produced by injecting ˜1×10⁶ human prostate cancer cell lines C4-2 into the right flank of an immune deficient nude mouse. After approximately 12 days, the tumors were at a size (diameter: 0.8-1.2 cm) that could be used for PAI experiments. Normalized PA intensity values were then calculated. The control mice that were injected with saline solution through the tail vein of the mice displayed only a 1.2-fold increase in the normalized PA signal at the tumor site after 24 h (FIG. 20). In contrast, injection of MMn (500 μM, 200 μL, 5 μmol/kg) through the tail vein of the mouse led to an overall average 3.1-fold increase in the normalized PA signal intensity at the tumor site after 24 h, relative to the pre-injection normalized PA signal intensity (FIG. 19). Without wishing to be bound by any theory, it is believed that the increase in PA signal was believed to be due to the gradual accumulation of MMn at the tumor, a phenomenon seen for porphyrinoid-based derivatives (Osterloh and Vicente 2002). In the case of MGd, an average 1.9-fold enhancement was seen (FIG. 21).

No significant increase in the PA signal at the tumor site was observed after the tail vein injection of ICG (500 μM, 200 μL 5 μmol/kg)—FIG. 22. This lack of enhancement is believed to be due to the rapid pharmacokinetics and liver-mediated clearance of ICG (Song et al., 2015; Qiu et al., 2020). While various nanoencapsulation strategies have been developed to overcome the limitations of ICG for in vivo applications, (Sheng et al., 2014; Chaudhary et al., 2019) MMn represents a single molecule that does not require such efforts to be able to directly image tumors in vivo.

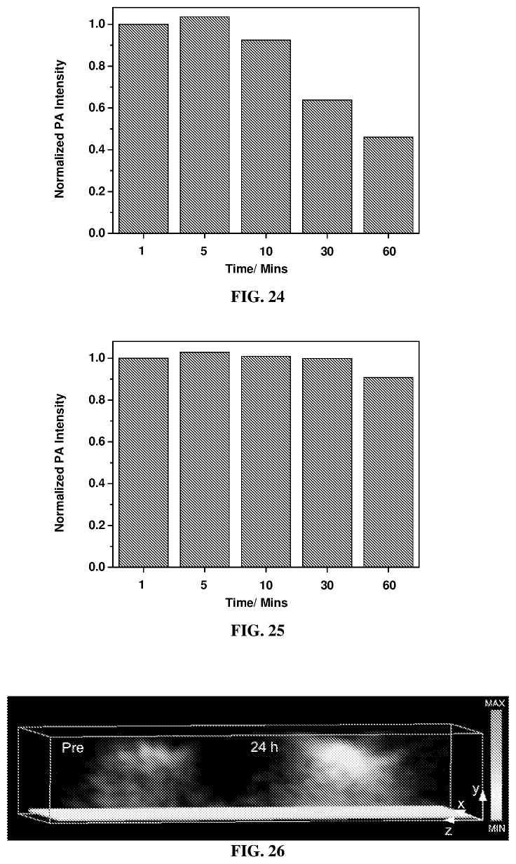

To confirm the in vivo stability of MMn for PA imaging, MMn was injected directly at the tumor site and the PA signal was measured over time (0-60 mins) with concurrent US imaging. ICG was used as a positive control. As seen in FIG. 23, an initial strong PA signal was observed for both ICG and MMn immediately after injection; however, as time progressed a significant decrease in the PA signal was observed in the case of ICG (46% of remaining PA signal—FIG. 24), whereas, the PA signal of MMn remained strong with minimal changes to the overall PA signal (91% of the PA signal remaining—FIG. 25).

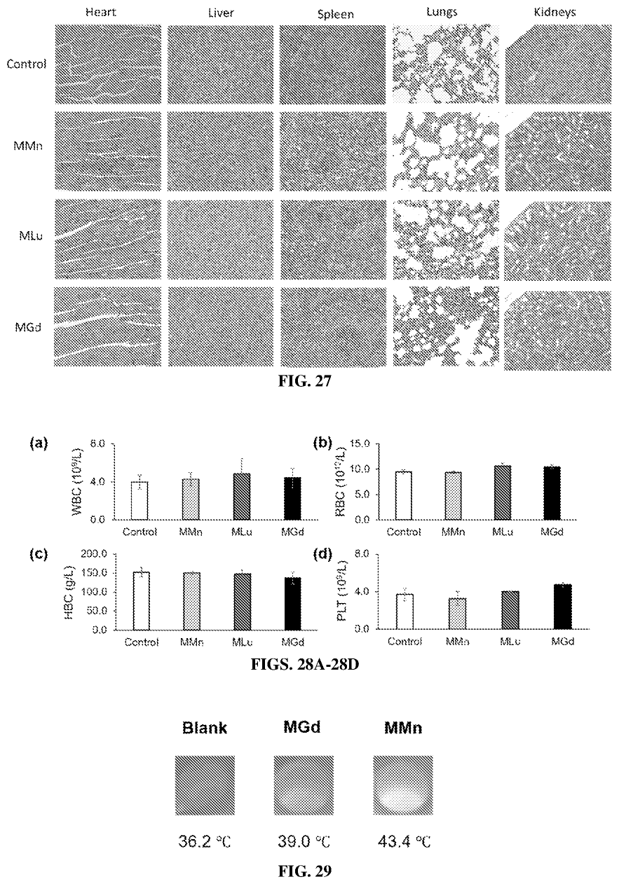

Using the PA imaging data obtained from MMn-treated tumor bearing mouse (injection concentration: 500 μM, 200 μL, (5 μmol/kg); tail vein administration), a 3D image of the tumor was constructed. As can be seen from an inspection of FIG. 26, a good image was obtained. Finally, to confirm the potential of MMn as a PA imaging agent, toxicity studies were carried out. As observed previously for MGd, (Khuntia 2007) no adverse toxicity to the major organs was observed in the MMn-treated mice, as inferred from Hematoxylin and Eosin (H&E) staining studies (FIG. 27). Moreover, essentially no changes to the overall blood cell counts (red blood cells (RBC), white blood cells (WBC), hemoglobin (HGB) and platelets (PLT)) complete blood count testing (Auto Hematology Analyzer, Mindray, BC-2800Vet) were seen in the case of mice treated with MMn, MGd or MLu (FIG. 28).

### Example 3: Photothermal Therapy with Manganese Compounds

### A. Methods and Materials

i. Photothermal Experiments of MMn and MMn Platinum Drug Conjugates

A549 cells (ATCC: CCL-185) were counted and seeded in transparent 96 well plates at a cell density of 14×10⁴/mL (100 μL in F12 medium (Gibco, Grand Island, NY, USA) with 10% fetal bovine serum (FBS, Gibco), penicillin and streptomycin (ps, 0.1%, Gibco)). Each plate was placed in the incubator at 37° C. and contained 5% CO₂ overnight. After 24 h, each experimental group was prepared using F12 medium (Gibco, Grand Island, NY, USA) with 10% fetal bovine serum (FBS, Gibco), penicillin and streptomycin (0.1%, Gibco) with blank (fill up with DMSO of 0.3%), MMn, MMn+HSA, Mono-MMn, Mono-MMn+HSA, Bis-MMn, Bis-MMn+HSA added. Additionally, MGd and MMn were tested. For the groups containing HSA, 10 μM of HSA was added to each corresponding solution and compound. The resulting solution was mixed for 10 minutes. The experimental groups were used and performed in triplicates. 100 μL of each solution was added to each well and then placed in the incubator for 4 h. For PTT experiments, each plate was then subjected to laser irradiation (Changchun New Industry Optoelectronic Technology Co., Ltd., MDL-N-808(FC)-10W, 808 nm laser wavelength, power: 1 W/cm²) for 15 minutes

ii. Cytotoxicity

A solution of MTS: PMS was prepared at a ratio of 20:1 in EP tube, and directly added to each well, (10 μL per well), placed in the incubator for 4 hours, and then the absorbance at 490 nm is measured by the Spectra Max multifunctional microplate reader (Molecular Devices, US) to calculate the cell survival rate.

### B. Photothermal Therapy of Manganese Derivatives

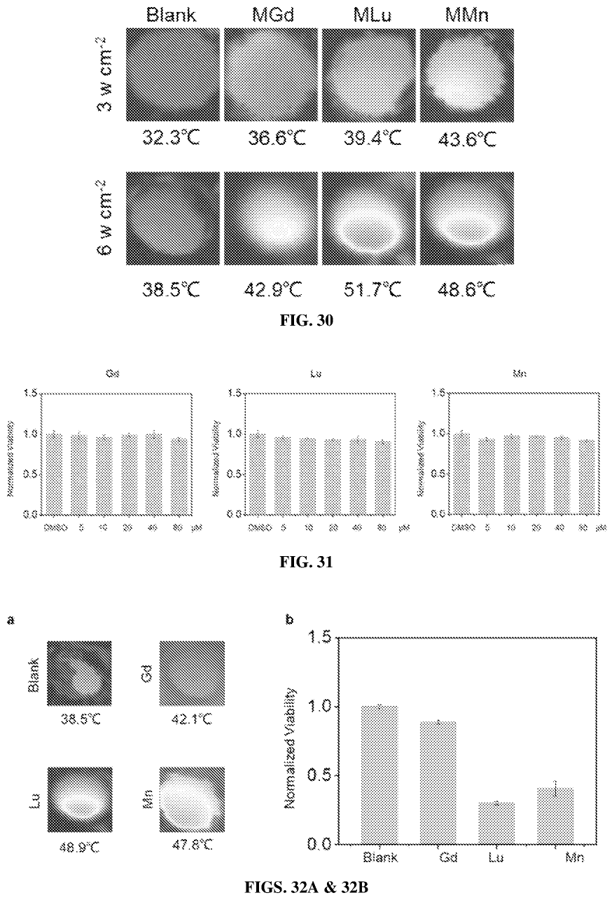

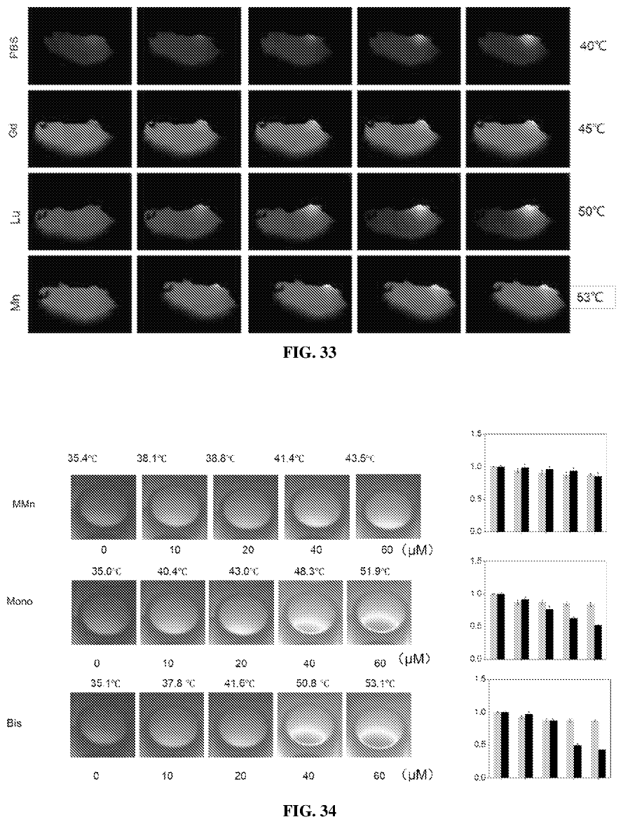

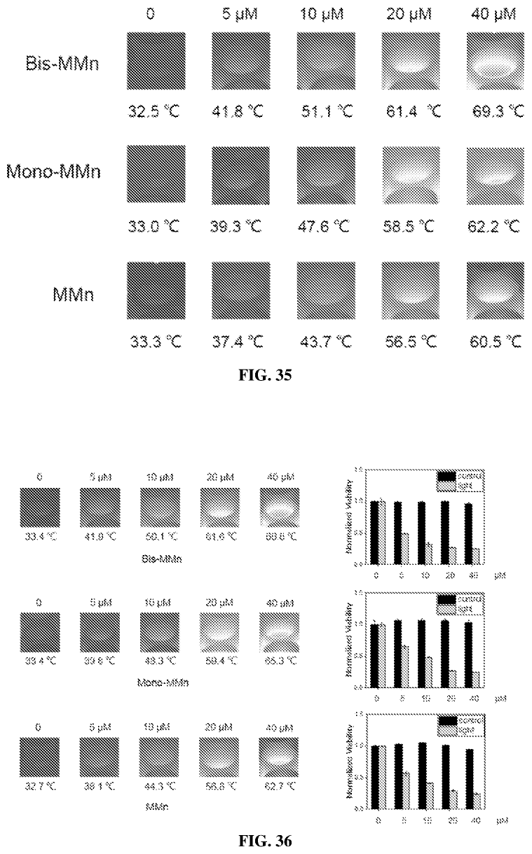

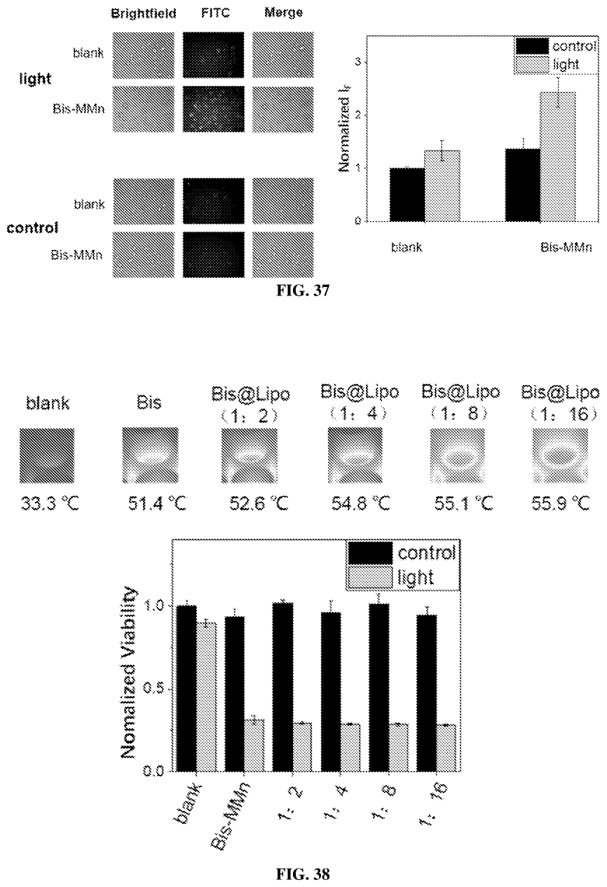

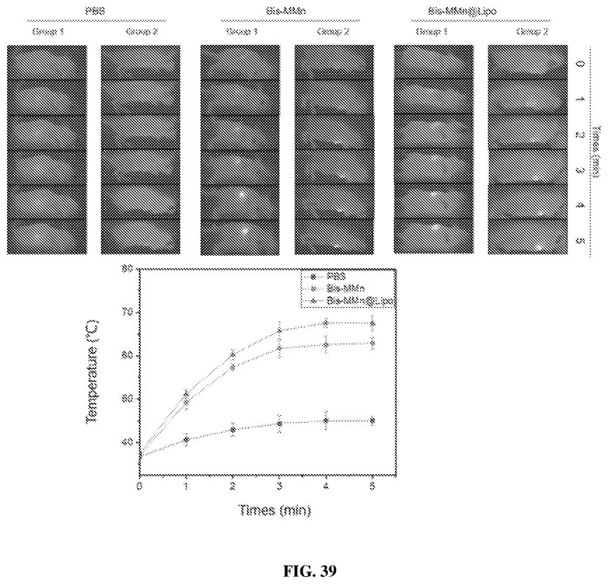

The photothermal effects of MMn were determined and compared to MGd. See FIG. 29. MGd showed an increase in temperature after irradiation of 2.8° C. compared to a blank while the MMn showed an increased temperature of 7.2° C., an increase of relatively 257% compared to MGd. Similarly, MMn, MLu, and MGd were all analyzed using two different light sources, a 3 W/cm² and 6 W/cm² at 808 nm for about 5 minutes per well at 40 μM. The 3 W/cm² showed little obvious change with MGd but both MLu and MMn showed increased temperatures but these temperatures did not reach 45° C. meaning that cells could be treated with prolonged irradiation. After the 6 W/cm² irradiation, each of the compounds showed increased temperature with the MLu and MMn showing particularly promise given the high temperatures reached. See, FIG. 30. As shown in FIG. 31, the cells remained viable without light after 24 hours. In FIG. 32, the MMn and MLu compounds after irradiation showed cell damage in MB-231 after irradiation. Similar results (FIG. 33) were obtained in MDA-MB-231 tumors in nu/nu mice. The photothermal effects of MMn along with mono and bis platinum(IV) derivates were analyzed in the presence and absence of human serum albumin (HSA). Relative to the blank, the non-platinated MMn showed an increased temperature in the absence of HSA of 4.1° C. and in the presence of HSA of 8.1° C. The addition of platinum groups showed an increase in the resultant temperature of 10.7° C. (absence of HSA) and 13.8° C. (presence of HSA) for the Mono-MMn derivative and 14.2° C. (absence of HSA) and 17.0° C. (presence of HSA) for the Bis-MMn. The increased heat from the photothermal effects of the compound showed a significant modulation in the cell viability of A549 and MDA-MB-231 cells compared to a blank when the compounds were exposed to light with less than 20% cell viability for the Bis-MMn. The cells were irradiated for 5 min per well with a light source at 808 nm at 3 W/cm². Increasing the number of Pt(IV) groups resulted in higher temperatures as the higher temperatures were obtained with Bis-MMn compared to Mono-MMn compared to MMn. See FIG. 34 and FIG. 35 for similar results in PBS solution. FIG. 36 shows the viability of MDA-MB-231 cells after irradiation time for 5 minutes per well using a 750 nm light source at 2 W.

Next, the generation of reactive oxygen species (ROS) with Bis-MMn was analyzed. After irradiation for 5 minutes per well with a 750 nm 2 W light source, the Bis-MMn showed the generation of ROS after irradiation with light. Samples that were not irradiated did not result in the generation of ROS.

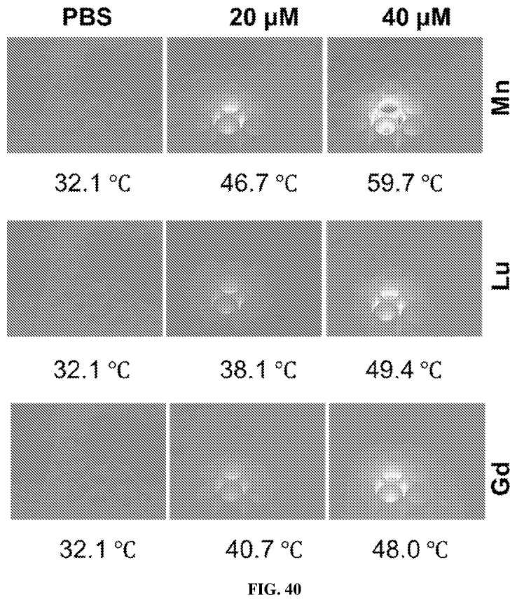

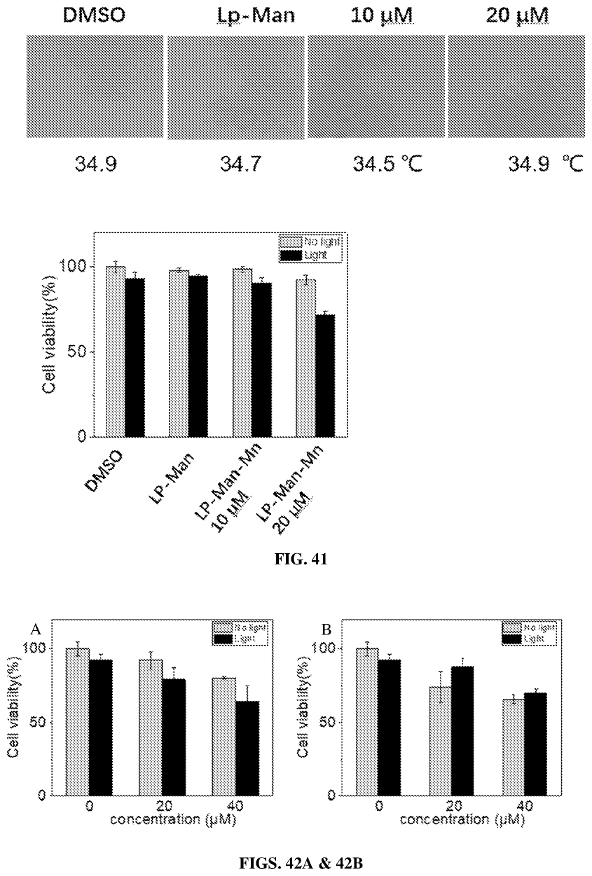

Finally, similar results were obtained when the compounds were formulated into liposomes. See. FIGS. 39-42.

All of the compositions and methods disclosed and claimed herein can be made and executed without undue experimentation in light of the present disclosure. While the compositions and methods of this invention have been described in terms of certain embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,935,498
U.S. Pat. No. 5,252,270
U.S. Pat. No. 5,272,142
U.S. Pat. No. 5,292,414
U.S. Pat. No. 5,369,101
U.S. Pat. No. 5,432,171
U.S. Pat. No. 5,439,570
U.S. Pat. No. 5,504,205
U.S. Pat. No. 5,533,354
U.S. Pat. No. 5,569,759
U.S. Pat. No. 5,583,220
U.S. Pat. No. 5,587,463
U.S. Pat. No. 5,591,422
U.S. Pat. No. 5,776,925
U.S. Pat. No. 5,955,586
U.S. Pat. No. 5,994,535
U.S. Pat. No. 6,207,660
U.S. Pat. No. 7,112,671
U.S. Pat. No. 8,410,263
U.S. Pat. No. 8,828,984
U.S. Pat. No. 10,406,167
Advanced Organic Chemistry: Reactions, Mechanisms, and Structure, March 2007.
Arambula et al., Anti-Cancer Agents in Medicinal Chemistry, 11, 222-232, 2011.
Arambula et al., Bioorg. Med. Chem. Lett., 21, 1701-1705, 2011.
Arambula et al., Dalton Trans., 48, 1 0834, 2009.
Arambula et al., Med. Chem. Commun., 3, 1275-1281, 2012.
Banfic' et al., Eur. J. Inorg. Chem., 484-492, 2014.
Barnes et al., et al., J. Chem. Biol., 11:557, 2004.
Berners-Price et al., Inorg. Chem., 33, 5842-5846, 1994.
Berners-Price et al., J. Am. Chem. Soc., 115, 8649-8659, 1993.
Bradley et al., Neuro-Oncol., 10(5):752-758, 2008.
Brewster et al., Chem., 6(3):703-724, 2020.
Borg and Rochford, Photochem. Photobiol., 94(6):1175-1209, 2018.
Chau et al., Exp. Cell Res., 241:269-272, 1998.
Chaudhary et al., Biomater. Sci., 7(12):5002-5015, 2019.
Choi et al., Inorg. Chem., 37, 2500-2504, 1998.
Choi et al., Inorg. Chem., 38, 1800-1805, 1999.
Davies et al., Inorg. Chem., 47:7673, 2008.
Dhar et al., J. Am. Chem. Soc., 131, 14652-14653, 2009.
Drougge and Elding, Inorg. Chim. Acta, 121:175, 1986.
Elding and Gustafson, Inorg. Chim. Acta, 19:165, 1976;
Feazell et al., J. Am. Chem. Soc., 129, 8438-8439, 2007.
Filippi et al., J. Biophotonics, 12(5), 2019.
Fischer et al., Neuro Toxicology, 29, 444-452, 2008.
Godwin et al., Proc. Natl. Acad. Sci. USA, 89, 3070-3074, 1992.
Guldi et al., J. Am. Chem. Soc., 122(34):8289-8298, 2000.
Guo et al., Adv. Mater., 31, 2019.
Handbook of Pharmaceutical Salts: Properties, and Use (P. H. Stahl & C. G. Wermuth eds., Verlag Helvetica Chimica Acta, 2002).
Hannah, et al., Org. Lett., 3(24):3911-3914, 2001.
Hannah et al., J. Am. Chem. Soc., 124(28):8416-8427, 2002.
Harrison and Zemp, IEEE International Ultrasonics Symposium Proceedings, 2357-2359, 2011.
Hashemy et al., J. Biol. Chem., 281(16):10691-10697, 2006.
He et al., Proc. Natl. Acad. Sci. USA, 97:5768-5772, 2000.
Huang et al., Proc. Natl. Acad. Sci. USA, 91:10394-10398, 1994.
Ines Batinic-Haberle, guest ed.; Bentham Science Publishers. DOI: 10.2 174/187 1520 11 795255894.
Johnstone et al., Inorg. Chem., DOI: 10.1021/ic400538c, 2013.
Keca et al., Angew. Chem. Intl. Ed., 55(21):6187-6191, 2016.
Keca et al., Advanced Healthcare Materials, 8(6), 2019.
Keca et al., Coord. Chem. Rev., 379:133-146, 2019.
Kelland, L. Nat Rev Cancer, 7:573-584, 2007.
Kim et al., Radiology, 255(2):442-450, 2010.
Kuroda et al., Inorg. Chem., 22, 3620-3624, 1983.
Lemma et al., Inorg. Chem., 39:1728, 2000.
Liu et al., Chem. Soc. Rev., 48(7):2053-2108, 2019.
Mindt et al., Photochem. Photobiol. Sci., 17(9):1189-1196, 2018.
Montagner et al., Angew. Chem. Int. Ed., 52,11785-11789, 2013.
Mosmann, T., J. Immunol. Meth., 65, 55-63, 1983.
Osterloh et al., J. Porphyr. Phthalocya., 6(5):305-324, 2002.
Pan et al., Crac. Channel: Methods and Protocols, 1843:55-62, 2018.
Philip et al., J. Photoch. Photobio., 96(1-3):137-148, 1996.
Qui et al., Biomed. Opt. Express, 11(7):3985-3995, 2020.
Ramalho et al., Am. J. Neuroradiol., 37(7):1192-1198, 2016.
Ramos et al., Quim. Nova, 34, 1450-1454, 2011.
Roat and Reedijk, J. Inorg. Bioch., 52, 263-274, 1993.
Rosenberg et al., Nature, 205:698, 1965.
Saxena et al., J. Photoch. Photobio. B, 74(1):29-38, 2004.
Sedgwick et al., Chem. Sci., 9(15):3672-3676, 2018.
Sedgwick et al., Chem. Soc. Rev., 49(10):2886-2915, 2020.
Senthilnithy et al., Luminescence, 24(4):203-208, 2009.
Seto et al., Toxicol. In Vitro, 34:113-119, 2016.
Siddik and Newman, Anal. Biochem., 172, 190-196, 1988.
Sheng et al., ACS Nano, 8(12):12310-12322, 2014.
Shimanovich, et al., J. Am. Chem. Soc., 123:3613-3614, 2001.
Siddik et al., Anal. Biochem., 163, 21-26; 1987.
Siddik et al., Cancer Res., 58, 698-703, 1998.
Sinisi et al., Inorg. Chem., 51, 9694-9704, 2012.
Song et al., RSC Adv., 5(5):3807-3813, 2015.
Thaibaud et al., Chem. —Eur. J., 20(29):8942-8947, 2014.
Thaibaud et al., Proc. Natl. Acad. Sci. U.S.A., 117(13):7021-7029, 2020.
Thomas and Khuntia, Int. J. Nanomedicine, 2(1):79-87, 2007.
Van der Veer et al., J. Inorg. Biochem., 26, 137-142, 1986.
Volchov et al., J. Fluoresc., 20(1):299-303, 2010.
Wang et al., J. Med. Chem., 61(19):8811-8824, 2018.
Weber et al., Nat. Methods, 13(8):639-650, 2016.
Wei et al., Org. Biomol. Chem., 3, 3290-3296, 2005.
Weigand et al., J. Phys. Chem., 101(42):7729-7734, 1997.
Wexselblatt et al., Angew. Chem. Int. Ed., 52, 6059-6062, 2013.
Wexselblatt et al., Inorg. Chim. Acta, 393, 75-83, 2012.
Xiao et al., Biomaterials, 32, 7732-7739, 2011.
Xiao et al., Chem. Commun., 48, 10730-10732, 2012.
Young et al., Proc. Natl. Acad. Sci. U.S.A., 93(13):6610-6615, 1996.
Zöllner et al., Mass Spectrom., 36, 742-753, 2001.

1. A compound of the formula: 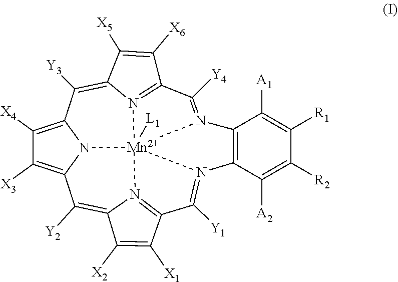
wherein:
R₁ and R₂ are each independently hydroxy, alkoxy_{(C≤12)}, substituted alkoxy_{(C≤12)}, 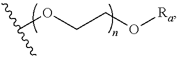
wherein n is 1-8 and Rₐ is hydrogen, alkyl_{(C≤6)}, or substituted alkyl_{(C≤6)}, or 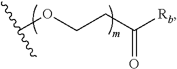
wherein m is 1-8 and R_{b} is hydroxy, alkoxy_{(C≤6)}, substituted alkoxy_{(C≤6)}, alkylamino_{(C≤6)}, substituted alkylamino_{(C≤6)}, dialkylamino_{(C≤6)}, substituted dialkylamino_{(C≤6)}, or a sugar moiety;
A₁ and A₂ are each hydrogen, halo, hydroxy, alkyl_{(C≤8)}, substituted alkyl_{(C≤8)}, aryl_{(C≤8)}, or substituted aryl_{(C≤8)};
Y₁, Y₂, Y₃, and Y₄ are each independently hydrogen, halo, hydroxy, alkyl_{(C≤8)}, or substituted alkyl_{(C≤8)};
X₁, X₂, X₃, X₄, X₅, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof, or a platinum(IV) chelating group; provided at least one of X₁-X₆ is a platinum(IV) chelating group, wherein the platinum(IV) chelating group is further defined as:
-A₃-Y₅-A₄-R_{c}
wherein:
A₃ and A₄ are each independently selected from alkanediyl_{(C≤8)}, substituted alkanediyl_{(C≤8)}, or 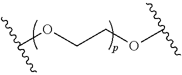
wherein p is 1-8;
Y₅ is —C(O)NR_{d}— or —NR_{d}C(O)—;
R_{d} is hydrogen, alkyl_{(C≤6)}, or substituted alkyl_{(C≤6)};
R_{c} is a group of the formula: 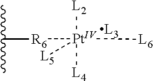
wherein:
R₆ is carboxy;
L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤18)};
L₆ is aqua, ammonia, nitrate, sulfate, halide, hydroxide, phosphate, or glucose-6-phosphate,
alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
L₁ is a monovalent anionic group;
or a pharmaceutically acceptable salt thereof.

2. The compound of claim 1 further defined as: 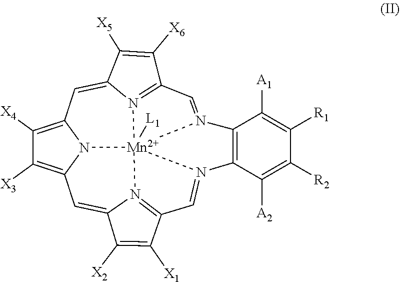
wherein:
R₁ and R₂ are each independently hydroxy, alkoxy_{(C≤12)}, substituted alkoxy_{(C≤12)}, 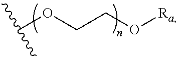
wherein n is 1-8 and Rₐ is hydrogen, alkyl_{(C≤6)}, or substituted alkyl_{(C≤6)}, or 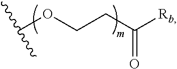
wherein m is 1-8 and R_{b} is hydroxy, alkoxy_{(C≤6)}, substituted alkoxy_{(C≤6)}, alkylamino_{(C≤6)}, substituted alkylamino_{(C≤6)}, dialkylamino_{(C≤6)}, substituted dialkylamino_{(C≤6)}, or a sugar moiety;
A₁ and A₂ are each hydrogen, halo, hydroxy, alkyl_{(C≤8)}, substituted alkyl_{(C≤8)}, aryl_{(C≤8)}, or substituted aryl_{(C≤8)};
X₁, X₂, X₃, X₄, X₅, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof, or a platinum(IV) chelating group; provided at least one of X₁-X₆ is a platinum(IV) chelating group, wherein the platinum(IV) chelating group is further defined as:
-A₃-Y₅-A₄-R_{c}
wherein:
A₃ and A₄ are each independently selected from alkanediyl_{(C≤8)},
substituted alkanediyl_{(C≤8)}, or 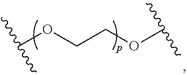
wherein p is 1-8;
Y₅ is —C(O)NR_{d}— or —NR_{d}C(O)—;
R_{d} is hydrogen, alkyl_{(C≤6)}, or substituted alkyl_{(C≤6)};
R_{c} is a group of the formula: 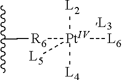
wherein:
R₆ is carboxy;
L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤18)};
L₆ is aqua, ammonia, nitrate, sulfate, halide, hydroxide, phosphate, or glucose-6-phosphate,
alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
L₁ is a monovalent anionic group;
or a pharmaceutically acceptable salt thereof.

3. The compound of claim 1 further defined as: 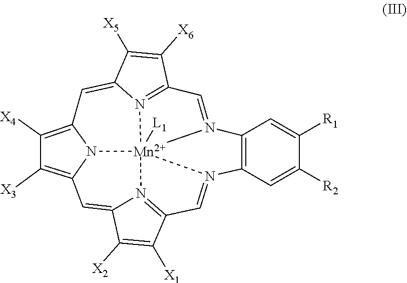
wherein:
R₁ and R₂ are each independently hydroxy, alkoxy_{(C≤12)}, substituted alkoxy_{(C≤12)}, 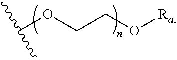
wherein n is 1-8 and Rₐ is hydrogen, alkyl_{(C≤6)}, or substituted alkyl_{(C≤6)}, or 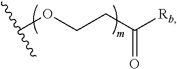
wherein m is 1-8 and R_{b} is hydroxy, alkoxy_{(C≤6)}, substituted alkoxy_{(C≤6)}, alkylamino_{(C≤6)}, substituted alkylamino_{(C≤6)}, dialkylamino_{(C≤6)}, substituted dialkylamino_{(C≤6)}, or a sugar moiety;
X₁, X₂, X₃, X₄, X₅, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof, or a platinum(IV) chelating group; provided at least one of X₁-X₆ is a platinum(IV) chelating group, wherein the platinum(IV) chelating group is further defined as:
-A₃-Y₅-A₄-R_{c}
wherein:
A₃ and A₄ are each independently selected from alkanediyl_{(C≤8)}, substituted alkanediyl_{(C≤8)}, or 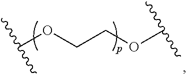
wherein p is 1-8;
Y₅ is —C(O)NR_{d}— or —NR_{d}C(O)—;
R_{d} is hydrogen, alkyl_{(C≤6)}, or substituted alkyl_{(C≤6)};
R_{c} is a group of the formula: 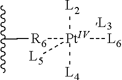
wherein:
R₆ is carboxy;
L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤18)};
L₆ is aqua, ammonia, nitrate, sulfate, halide, hydroxide, phosphate, or glucose-6-phosphate,
alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
L₁ is a monovalent anionic group;
or a pharmaceutically acceptable salt thereof.

4. The compound of claim 1 further defined as: 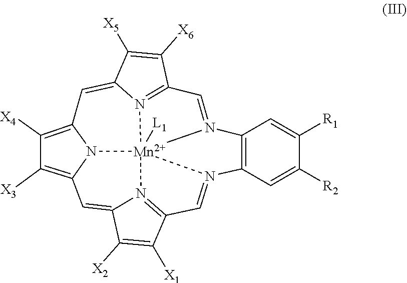
wherein:
R₁ and R₂ are each independently hydroxy, alkoxy_{(C≤12)}, substituted alkoxy_{(C≤12)}, 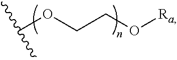
wherein n is 1-8 and Rₐ is hydrogen, alkyl_{(C≤6)}, or substituted alkyl_{(C≤6)};
X₁, X₂, X₃, X₄, X₅, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof, or a platinum(IV) chelating group; provided at least one of X₁-X₆ is a platinum(IV) chelating group, wherein the platinum(IV) chelating group is further defined as:
-A₃-Y₅-A₄-R_{c}
wherein:
A₃ and A₄ are each independently selected from alkanediyl_{(C≤8)},
substituted alkanediyl_{(C≤8)}, or 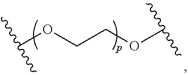
wherein p is 1-8;
Y₅ is —C(O)NR_{d}— or —NR_{d}C(O)—;
R_{d} is hydrogen, alkyl_{(C≤6)}, or substituted alkyl_{(C≤6)};
R_{c} is a group of the formula: 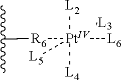
wherein:
R₆ is carboxy;
L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤18)};
L₆ is aqua, ammonia, nitrate, sulfate, halide, hydroxide, phosphate, or glucose-6-phosphate,
alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
L₁ is a monovalent anionic group;
or a pharmaceutically acceptable salt thereof.

5. The compound of claim 1 further defined as: 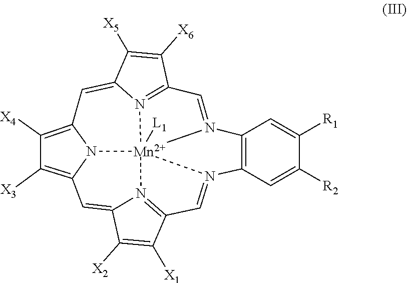
wherein:
R₁ and R₂ are each independently hydroxy, alkoxy_{(C≤12)}, substituted alkoxy_{(C≤12)}, 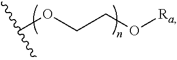
wherein n is 1-8 and Rₐ is hydrogen, alkyl_{(C≤6)}, or substituted alkyl_{(C≤6)};
X₁, X₃, X₄, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof;
X₂ and X₅ are each independently alkyl_{(C≤8)}, substituted alkyl_{(C≤8)}, a platinum(IV) chelating group; provided either X₂ or X₅ is a platinum(IV) chelating group, wherein the platinum(IV) chelating group is further defined as:
-A₃-Y₅-A₄-R_{c}
wherein:
A₃ and A₄ are each independently selected from alkanediyl_{(C≤8)},
substituted alkanediyl_{(C≤8)}, or 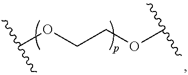
wherein p is 1-8;
Y₅ is —C(O)NR_{d}— or —NR_{d}C(O)—;
R_{d} is hydrogen, alkyl_{(C≤6)}, or substituted alkyl_{(C≤6)};
R_{c} is a group of the formula: 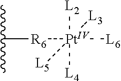
wherein:
R₆ is carboxy;
L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤18)};
L₆ is aqua, ammonia, nitrate, sulfate, halide, hydroxide, phosphate, or glucose-6-phosphate,
alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups; and
L₁ is a monovalent anionic group;
or a pharmaceutically acceptable salt thereof.

6. The compound of claim 1 further defined as: 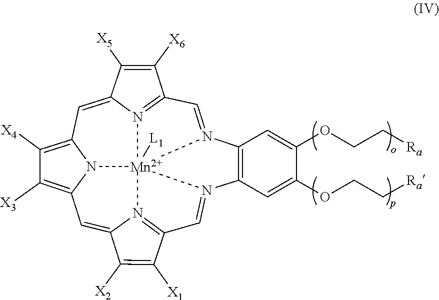
wherein:
Rₐ and Rₐ′ are each independently hydrogen, alkyl_{(C≤6)}, or substituted alkyl_{(C≤6)};
o and p are each independent 1, 2, 3, or 4;
X₁, X₃, X₄, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof;
X₂ and X₅ are each independently alkyl_{(C≤8)}, substituted alkyl_{(C≤8)}, a platinum(IV) chelating group; provided either X₂ or X₅ is a platinum(IV) chelating group, wherein the platinum(IV) chelating group is further defined as:
-A₃-Y₅-A₄-R_{c}
wherein:
A₃ and A₄ are each independently selected from alkanediyl_{(C≤8)},
substituted alkanediyl_{(C≤8)}, or 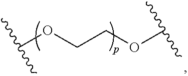
wherein p is 1-8;
Y₅ is —C(O)NR_{d}— or —NR_{d}C(O)—;
R_{d} is hydrogen, alkyl_{(C≤6)}, or substituted alkyl_{(C≤6)};
R_{c} is a group of the formula: 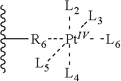
wherein:
R₆ is carboxy;
L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤18)};
L₆ is aqua, ammonia, nitrate, sulfate, halide, hydroxide, phosphate, or glucose-6-phosphate,
alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups; and
L₁ is a monovalent anionic group;
or a pharmaceutically acceptable salt thereof.

7.-26. (canceled)

27. The compound of claim 1,
wherein X₁, X₃, X₄, X₅, and X₆ are each alkyl_{(C≤8)} or substituted alkyl_{(C≤8)}.

28. (canceled)

29. (canceled)

30. The compound of claim 1,
wherein X₂ is a platinum(IV) chelating group.

31. The compound of claim 1,
wherein A₃ is alkanediyl_{(C≤8)}, Y₅ is —NR_{d}C(O)—, and A₄ is alkanediyl_{(C≤8)}.

32.-38. (canceled)

39. The compound of claim 1, wherein L₂ is halide, or ammonia, or L₂ and L₃ are taken together and are diaminocycloalkane_{(C≤18)} or substituted diaminocycloalkane_{(C≤18)}, or L₂ and L₃ are taken together and are alkyldicarboxylate_{(C≤18)} or substituted alkyldicarboxylate_{(C≤18)}, or L₃ is halide or ammonia, or L₄ is halide or ammonia, or L₄ and L₅ are taken together and are diaminocycloalkane_{(C≤18)} or substituted diaminocycloalkane_{(C≤18)}, or L₄ and L₅ are taken together and are alkyldicarboxylate_{(C≤18)} or substituted alkyldicarboxylate_{(C≤18)}, or L₅ is halide or ammonia.

40.-62. (canceled)

63. The compound of claim 1, wherein L₆ is hydroxy, alkylcarboxylate_{(C≤12)}, substituted alkylcarboxylate_{(C≤12)}, or halo.

64.-77. (canceled)

78. The compound of claim 1, wherein X₅ is a platinum(IV) chelating group.

79.-120. (canceled)

121. The compound of claim 1, wherein L₁ is nitrate, alkylcarboxylate_{(C≤12)}, or substituted alkylcarboxylate_{(C≤12)}.

122.-124. (canceled)

125. The compound of claim 1 further defined as: 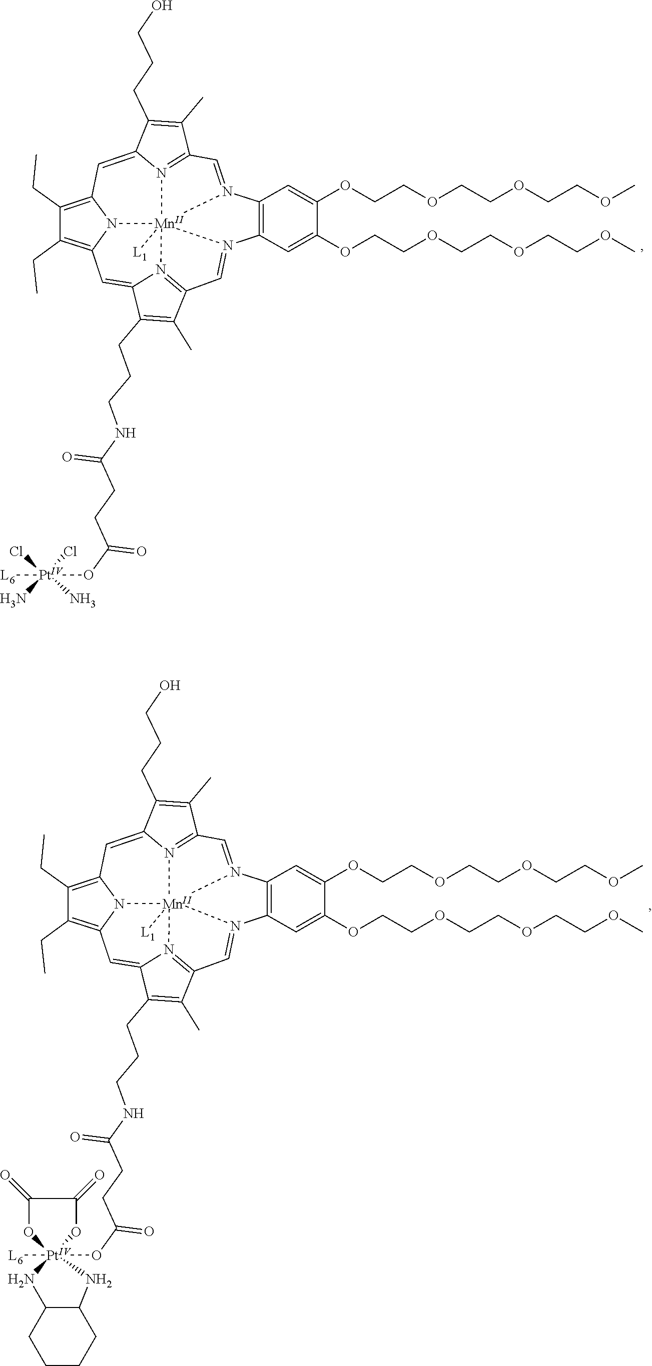 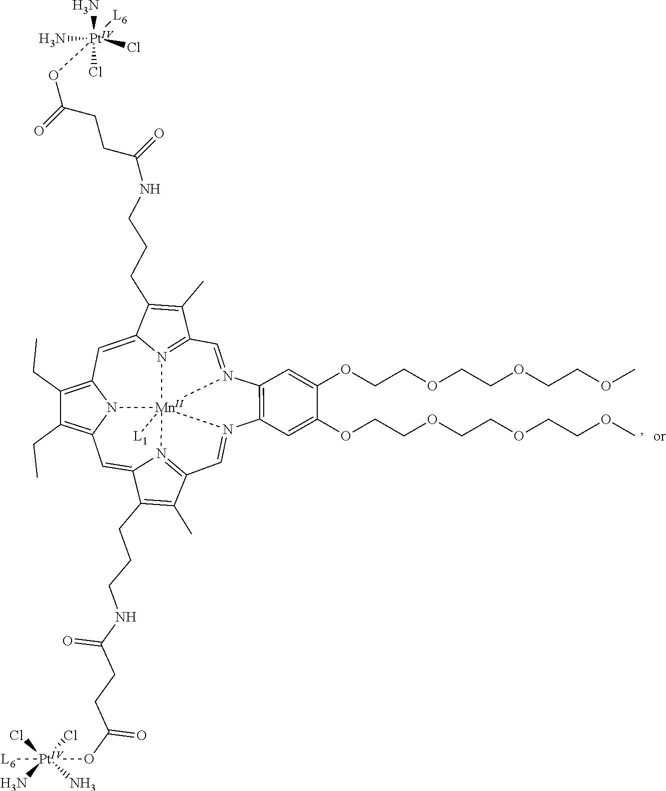 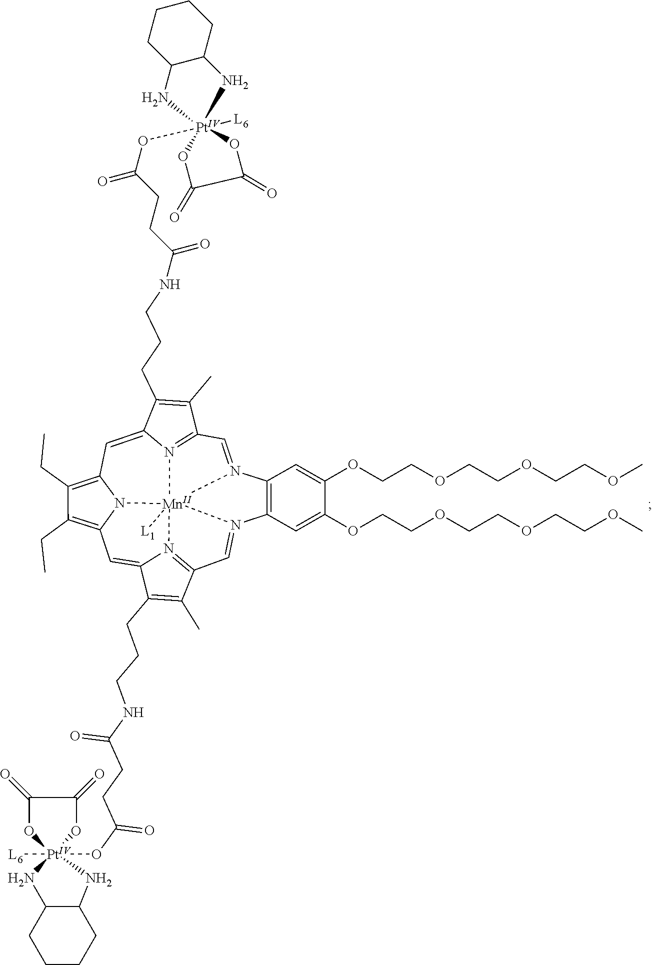
wherein:
L₁ is a monovalent anionic group; and
each L₆ is aqua, ammonia, nitrate, sulfate, halide, hydroxide, phosphate, or glucose-6-phosphate,
alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
or a pharmaceutically acceptable salt thereof.

126. The compound of claim 125 further defined as: 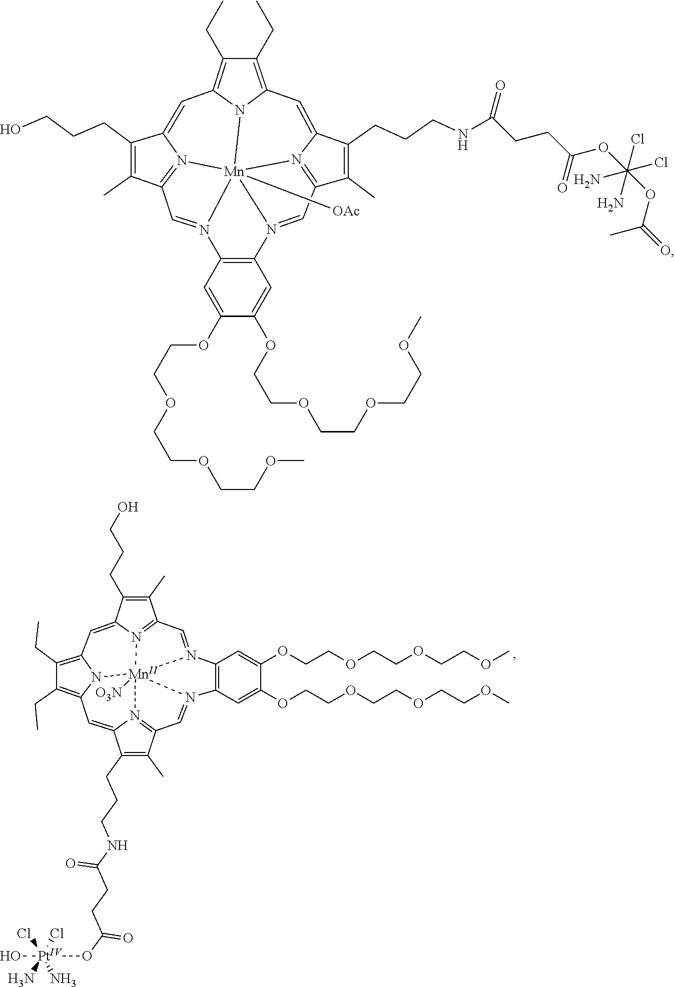 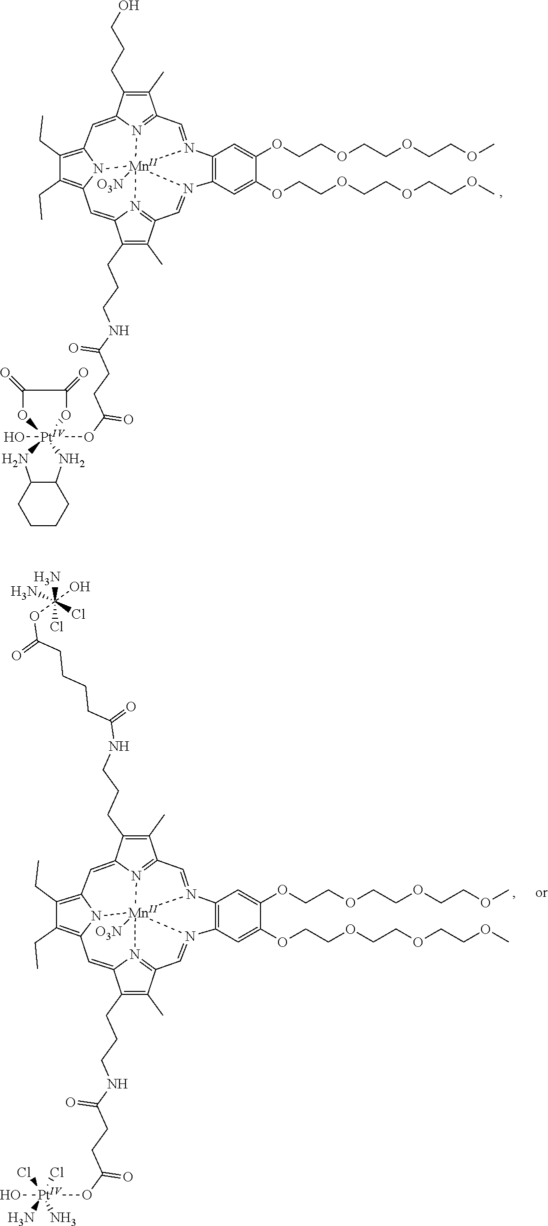 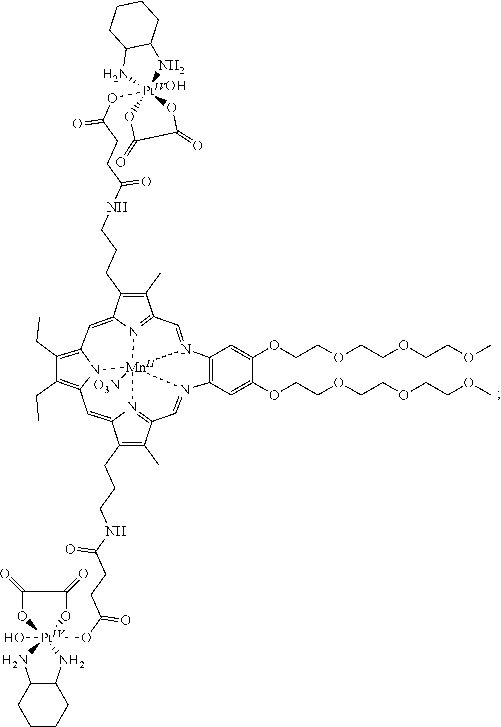
or a pharmaceutically acceptable salt thereof.

127. A pharmaceutical composition comprising:
(A) a compound of claim 1; and
(B) an excipient.

128.-131. (canceled)

132. A method of treating a disease comprising administering a therapeutically effective amount of a compound of claim 1 to a patient in need thereof.

133. The method of claim 132, wherein the disease is cancer.

134.-141. (canceled)

142. A method of obtaining an image of a patient comprising administering to the patient an effective amount of a compound or a pharmaceutical composition comprising a compound of the formula: 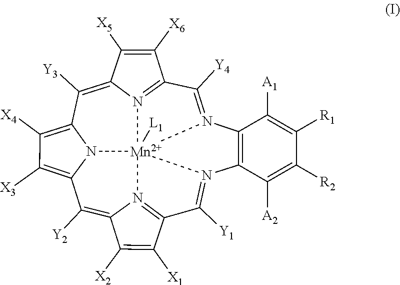
wherein:
R₁ and R₂ are each independently hydroxy, alkoxy_{(C≤12)}, substituted alkoxy_{(C≤12)}, 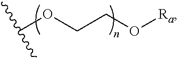
wherein n is 1-8 and Rₐ is hydrogen, alkyl_{(C≤6)}, or substituted alkyl_{(C≤6)}, or 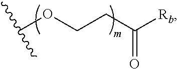
wherein m is 1-8 and R_{b} is hydroxy, alkoxy_{(C≤6)}, substituted alkoxy_{(C≤6)}, alkylamino_{(C≤6)}, substituted alkylamino_{(C≤6)}, dialkylamino_{(C≤6)}, substituted dialkylamino_{(C≤6)}, or a sugar moiety;
A₁ and A₂ are each hydrogen, halo, hydroxy, alkyl_{(C≤8)}, substituted alkyl_{(C≤8)}, aryl_{(C≤8)}, or substituted aryl_{(C≤8)};
Y₁, Y₂, Y₃, and Y₄ are each independently hydrogen, halo, hydroxy, alkyl_{(C≤8)}, or substituted alkyl_{(C≤8)};
X₁, X₂, X₃, X₄, X₅, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof, or a platinum(IV) chelating group; wherein the platinum(IV) chelating group is further defined as:
-A₃-Y₅-A₄-R_{c}
wherein:
A₃ and A₄ are each independently selected from alkanediyl_{(C≤8)},
substituted alkanediyl_{(C≤8)}, or 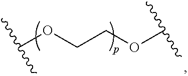
wherein p is 1-8;
Y₅ is —C(O)NR_{d}— or —NR_{d}C(O)—;
R_{d} is hydrogen, alkyl_{(C≤6)}, or substituted alkyl_{(C≤6)};
R_{c} is a group of the formula: 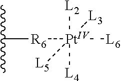
wherein:
R₆ is carboxy;
L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤18)};
L₆ is aqua, ammonia, nitrate, sulfate, halide, hydroxide, phosphate, or glucose-6-phosphate,
alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
L₁ is a monovalent anionic group;
and imaging the patient to obtain the image of the patient.

143.-163. (canceled)

164. A method of treating a patient comprising administering a compound of the formula: 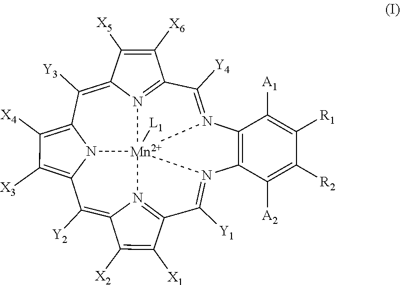
wherein:
R₁ and R₂ are each independently hydroxy, alkoxy_{(C≤12)}, substituted alkoxy_{(C≤12)}, 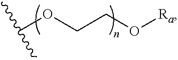
wherein n is 1-8 and Rₐ is hydrogen, alkyl_{(C≤6)}, or substituted alkyl_{(C≤6)}, or 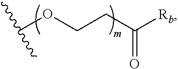
wherein m is 1-8 and R_{b} is hydroxy, alkoxy_{(C≤6)}, substituted alkoxy_{(C≤6)}, alkylamino_{(C≤6)}, substituted alkylamino_{(C≤6)}, dialkylamino_{(C≤6)}, substituted dialkylamino_{(C≤6)}, or a sugar moiety;
A₁ and A₂ are each hydrogen, halo, hydroxy, alkyl_{(C≤8)}, substituted alkyl_{(C≤8)}, aryl_{(C≤8)}, or substituted aryl_{(C≤8)};
Y₁, Y₂, Y₃, and Y₄ are each independently hydrogen, halo, hydroxy, alkyl_{(C≤8)}, or substituted alkyl_{(C≤8)};
X₁, X₂, X₃, X₄, X₅, and X₆ are each independently hydrogen, alkyl_{(C≤8)}, cycloalkyl_{(C≤8)}, alkenyl_{(C≤8)}, alkynyl_{(C≤8)}, aryl_{(C≤8)}, heteroaryl_{(C≤8)}, heterocycloalkyl_{(C≤8)}, or a substituted version thereof, or a platinum(IV) chelating group; wherein the platinum(IV) chelating group is further defined as:
-A₃-Y₅-A₄-R_{c}
wherein:
A₃ and A₄ are each independently selected from alkanediyl_{(C≤8)},
substituted alkanediyl_{(C≤8)}, or 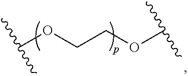
wherein p is 1-8;
Y₅ is —C(O)NR_{d}— or —NR_{d}C(O)—;
R_{d} is hydrogen, alkyl_{(C≤6)}, or substituted alkyl_{(C≤6)};
R_{c} is a group of the formula: 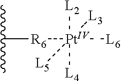
wherein:
R₆ is carboxy;
L₂-L₅ are each independently selected or two or more may be taken together from ammonia, halide, diaminocycloalkane_{(C≤12)}, substituted diaminocycloalkane_{(C≤12)}, alkyldicarboxylate_{(C≤18)}, or substituted alkyldicarboxylate_{(C≤18)};
L₆ is aqua, ammonia, nitrate, sulfate, halide, hydroxide, phosphate, or glucose-6-phosphate,
alkylamine_{(C≤12)}, cycloalkylamine_{(C≤12)}, dialkylamino_{(C≤18)}, dicycloalkylamine_{(C≤18)}, arylamine_{(C≤12)}, diarylamine_{(C≤18)}, diaminoalkane_{(C≤12)}, diaminocycloalkane_{(C≤12)}, diaminoarene_{(C≤12)}, heteroarene_{(C≤12)}, alkylcarboxylate_{(C≤12)}, alkyldicarboxylate_{(C≤18)}, arylcarboxylate_{(C≤12)}, aryldicarboxylate_{(C≤18)}, or a substituted version of any of these groups;
L₁ is a monovalent anionic group;
to a patient in need thereof and exposing the patient to an electromagnetic radiation.

165.-179. (canceled)
